(19) United States
(12) Patent Application Publication
(10) Pub. No.: US 2024/0005009 A1
(43) Pub. Date: Jan. 4, 2024

(54) APPARATUS AND METHOD FOR CONSENT CONTROLLED HEALTH RECORD ACCESS

(71) Applicant: Medinex Corp., Palmdale, CA (US)

(72) Inventors: Sanjaya Khanal, Lancaster, CA (US); Abdallah Farrukh, Palmdale, CA (US); Augie L. Vasic, Olathe, KS (US); Sagun Rayamajhi, Northridge, CA (US); Sandeep Puri, San Jose, CA (US)

(21) Appl. No.: 18/035,287

(22) PCT Filed: Nov. 13, 2020
(86) PCT No.: PCT/US2020/060615

§ 371 Date: May 3, 2023

(51) Int. Cl. G06F 21/60; G06F 21/62; G16H 10/60

(52) U.S. Cl. CPC G06F 21/602; G06F 21/6209; G16H 10/60

(57) ABSTRACT

Apparatus and associated methods relate to configuring secure access to a patient's stored health record data, in response to receiving the patient's consent to a health record data access request, and providing the secure access to a specific user at a location and time related to the request. The access request may be received from a provider. The provider may be a doctor. Secure access by a specific user to a discrete health record data field may be configured, granted, or revoked based on patient consent status, permitting the patient selective control over access to their personal health record data. Access encryption parameters distinct from the storage encryption parameters securing the stored health record may be configured to permit specific user access to a specific data field. The access encryption parameters may be revoked by the patient withdrawing consent, permitting the patient to assert ownership control of their health records.

### RELATED APPLICATIONS

None.

### TECHNICAL FIELD

This disclosure relates generally to health record access.

### BACKGROUND

Patient health records are health data. A patient's health data are a record of the patient's health. Patient health records document patient health by encoding patient medical data. Patient medical data may include historical and current patient medical status. Patient medical data may be, for example, test results or treatment details. A patient may be sensitive about some medical data included in their health records. Various regulations may govern health record access, to protect patient privacy.

Health records may be encoded in various forms. Some health records may be paper records. Various health records may be digitally rendered in visual form. In an illustrative example, health records may be encoded digital data. Users of health records include individuals, organizations, computer applications, and electronic devices. Users may employ health records to aid patient health evaluation, diagnosis, or treatment. For example, a doctor may study a patient's health records to determine effective treatment for the patient, based on the patient's health history documented by the health records.

A provider such as a doctor may generate new medical record data related to a patient, as a result of treating or examining the patient. Access to a patient's health records may be limited to users with permission. For example, a provider needing access to a patient's health records may need to request permission to access the records. A patient may need to approve provider access to the patient's records. Patients and providers may expend substantial effort managing access to patient health records.

### SUMMARY

Apparatus and associated methods relate to configuring secure access to a patient's stored health record data, in response to receiving the patient's consent to a health record data access request, and providing the secure access to a specific user at a location and time related to the request. The access request may be received from a provider. The provider may be a doctor. Secure access by a specific user to a discrete health record data field may be configured, granted, or revoked based on patient consent status, permitting the patient selective control over access to their personal health record data. Access encryption parameters distinct from the storage encryption parameters securing the stored health record may be configured to permit specific user access to a specific data field. The access encryption parameters may be revoked by the patient withdrawing consent, permitting the patient to assert ownership control of their health records.

In an aspect, an apparatus is disclosed, comprising: a processor; and, a memory, operably coupled with the processor, wherein the memory encodes processor executable program instructions and data to program and configure the processor to cause the apparatus to perform operations comprising: store a patient's health record data encrypted with storage encryption parameters; in response to the patient giving consent to a data access request: configure secure access to the patient's health record data based on encrypting the stored data with access encryption parameters distinct from the storage encryption parameters; and, provide the secure access to a specific device at a location and time related to the request.

The data access request may further comprise a data upload operation request.

The data access request may further comprise a data download operation request.

The data access request may further comprise a data share operation request.

The data access request may further comprise a data delete operation request.

Provide the secure access may further comprise implement the data operation request.

Store the patient's health record data may further comprise separate the health record data into a plurality of data fields based on the data type.

Store the patient health record data may further comprise encrypt each data field of the plurality of data fields with a storage encryption parameter comprising an encryption key uniquely determined as a function of information related to the patient.

Configure secure access may further comprise configure distinct access encryption parameters for each data field of the plurality of data fields.

The access encryption parameters may further comprise a decryption key uniquely determined as a function of an individual data field.

The access encryption parameters may further comprise a decryption key uniquely determined as a function of the data access request.

Provide the secure access may further comprise sending a digital message comprising the access encryption parameters.

The digital message may further comprise an encrypted reference to the decryption key.

Provide the secure access may further comprise send a digital message comprising a response to the data access request.

The response may further comprise consent to the data access request.

The response may further comprise denial to the data access request.

The response may be determined as a function of the data access requestor's identity authenticated based on sensor data.

The response may be determined as a function of the data access requestor's location authenticated based on sensor data.

The operations performed by the processor may further comprise in response to a patient withdrawing consent to data access, revoking the configured access to the data.

The operations performed by the processor may further comprise in response to a patient giving consent to a data access request, creating a provider encounter data bundle.

The provider encounter data bundle structure may be designed to permit individual access to patient data fields and records.

In another aspect, an apparatus is disclosed, comprising: a processor; a cloud database, operably coupled with the processor; and, a memory, operably coupled with the processor, wherein the memory encodes processor executable program instructions and data to program and configure the processor to cause the apparatus to perform operations comprising: receive a digital message comprising a request by a provider to upload a patient's health record data; send a digital message comprising a request for the patient's consent for the provider to upload the health record data; in response to receiving a digital message comprising the patient's consent to upload the health record data: send a digital message to the provider comprising consent to upload the health record data; receive a digital message comprising the health record data; separate the health record data into a plurality of data fields based on the data type determined for each data field of the plurality of data fields; encrypt each data field of the plurality of data fields based on storage encryption parameters comprising an encryption key uniquely determined as a function of account information associated to the patient; and, store the encrypted health record data to the cloud database.

Separate the health record data may further comprise digitally rendering the health record data based on rendering the health record data to a file.

Separate the health record data may further comprise extract to digital form the data encoded by the rendered health record.

Separate the health record data may further comprise the data type determined by an AI (Artificial Intelligence) process configured to recognize the type of data encoded by a health record data field.

The digital message comprising the request for the patient's consent may further comprise the provider location authenticated based on sensor data received from the provider.

The digital message comprising the request for the patient's consent may further comprise the provider device authenticated based on a communication interface parameter received from the provider.

The operations performed by the processor may further comprise in response to receiving the digital message comprising the patient's consent, creating a provider encounter data bundle.

The provider encounter data bundle structure may be designed to permit individual access to patient data fields and records.

In another aspect, an apparatus is disclosed, comprising: a processor; a cloud database, operably coupled with the processor; and, a memory, operably coupled with the processor, wherein the memory encodes processor executable program instructions and data to program and configure the processor to cause the apparatus to perform operations comprising: receive a digital message comprising a request by a sending provider to share a patient's health record data with a receiving provider; send a digital message comprising a request for the patient's consent for the sending provider to share the patient's health record data with the receiving provider; in response to receiving a digital message comprising the patient's consent to share the health record data: send a digital message to the sending provider comprising consent to share the health record data; and, receive a digital message from the receiving provider comprising an acknowledgement the shared health record data has been received by the receiving provider.

The operations performed by the processor may further comprise send to the sending provider a digital message comprising a unique session key configured to secure the health record data in transit to the receiving provider.

The operations performed by the processor may further comprise send to the receiving provider a digital message comprising the health record data to be shared with the receiving provider.

The operations performed by the processor may further comprise configure secure access to the shared data for the receiving provider based on encrypting the stored data with access encryption parameters determined as a function of the share request.

The operations performed by the processor may further comprise provide the secure access to the receiving provider at a location and time related to the request.

The secure access to the receiving provider at a location and time related to the request may further comprise the receiving provider location determined as a function of sensor data.

The sensor data may be GPS data.

Provide the secure access to the receiving provider at a location and time related to the request may further comprise configuring the access encryption parameters to expire after a predetermined access time period has passed.

The operations performed by the processor may further comprise in response to determining the predetermined access time period passed, revoke the access encryption parameters.

Provide the secure access to the receiving provider at a location and time related to the request may further comprise in response to determining the receiving provider location changed from a predetermined location, revoke the access encryption parameters.

The operations performed by the processor may further comprise in response to determining the receiving provider location returned to the predetermined location, restore the access encryption parameters.

Determining the receiving provider location changed from a predetermined location may further comprise determining the location changed by a distance greater than or equal to a predetermined minimum radius from the predetermined location.

Determining the receiving provider location changed from a predetermined location may further comprise determining the location changed for a period of time greater than or equal to a predetermined minimum period of time.

In another aspect, an apparatus is disclosed, comprising: a processor; and, a memory, operably coupled with the processor, wherein the memory encodes processor executable program instructions and data to program and configure the processor to cause the apparatus to perform operations comprising: in response to receiving a request to download a patient's stored health record data encrypted with storage encryption parameters: determine if the patient has given consent for the requestor to download the patient's health record data; in response to determining the patient has given consent: configure secure access by the requestor to the patient's health record data, based on providing the requestor access encryption parameters distinct from the storage encryption parameters; and, provide the secure access to the requestor for a specific device at a location and time related to the request; and, in response to determining the patient has not given consent: deny the request.

The apparatus may further comprise a cloud database operably coupled with the processor, and provide the secure access may further comprise provide the secure access to the health record data by the database.

Receiving the request to download the patient's health record data may further comprise receiving a digital message comprising the request.

Determine if the patient has given consent may further comprise determine if the patient's consent is predetermined for the requestor.

Determine if the patient has given consent may further comprise receive a digital message comprising the patient's consent.

The health record data may further comprise a plurality of data fields.

The access parameters may further comprise an encrypted reference to a unique session key configured to secure the health record data in transit.

Provide the secure access to the requestor for a specific device at a location and time related to the request may further comprise the device authenticated based on a security certificate.

Provide the secure access to the requestor for a specific device at a location and time related to the request may further comprise the location determined based on sensor data.

Provide the secure access to the requestor for a specific device at a location and time related to the request may further comprise the access time limited based on the access parameters configured to expire at a predetermined time.

Deny the request may further comprise send a digital message comprising request denial.

Various implementations may achieve one or more technical effect. For example, some implementations may improve a user's ease of managing access to health records. This facilitation may be a result of reducing the user's effort granting or denying health care providers access to the user's health records. In some implementations, secure portable access to a user's health records may be automatically configured for a provider new to a user, in response to the user's consent. Such automatic secure portable shared access to the user's health records by a new provider may reduce a user's risk of exposure to unauthorized access to the user's private sensitive medical data, while reducing the user's effort to share health data with a new provider authorized by the user. Some implementations may reduce the effort required to control a provider's access to a user's health record data when the patient decides the provider should no longer have access. Such patient ownership control of health record data may be a result of secure health record access based on encryption parameters that may be revoked when patient consent is withdrawn.

Some implementations may improve patient privacy protection. This facilitation may be a result of configuring secure access to a discrete health record data field, to permit a user to access only the specific portions of the health record the patient decides the user should be allowed to access. Various implementations may improve the efficiency of managing patient health record access. Such improved health record access management efficiency may be a result of configuring secure access to health record data by a specific user at a specific location for a predetermined period of time, and automatically revoking the access when the time period has passed. For example, a patient may provide consent for a doctor or insurer to access their health record temporarily, and automatically revoke access when the need to access the health record expires, reducing the potential for unauthorized possession of the patient's health records.

In an exemplary implementation, a patient may register to secure their personal health record data by authorizing parameters governing encryption to protect the stored data. The personal health record data may be received from the patient or a health care provider. The patient may assert ownership control over their personal health record data based on secure storage, access, or sharing governed by patient consent. For example, the patient may provide consent for a provider, such as, for example, a doctor, or hospital, to upload patient medical data generated by the provider, thereby adding to the health record data controlled by the patient. The patient may provide consent for a provider to share health record data with another provider. Access to the health record data may be provided based on patient consent to access the data. Access to the health record data may be revoked based on a change in the patient's consent.

Received health record data may be captured from a health record based on digitally rendering the record in visual form, and processing the rendered health record to capture the data. The health record may be digitally rendered to a file. The data may be captured from the digitally rendered file based on processing the file with a file data extraction process configured to harvest data fields from the file. The file data extraction process may be, for example, a data extraction utility program. The received personal health record data may be transformed to distinct fields encoding data of various types, permitting storage access to the individual data fields to be separately controlled, based on the data type. For example, access to a data field encoding patient identifying information may be controlled separately from a data field encoding medical test result data.

The personal health record data may be stored encrypted with storage encryption parameters governed by the patient to facilitate patient control and ownership of the data. The patient may consent to a request for access to the patient's stored encrypted personal health data. When the patient provides consent to access the stored encrypted data, secure access to the data may be configured based on encrypting the data with access encryption parameters distinct from the storage encryption parameters.

The access encryption parameters may include an ephemeral encryption key valid only during a session for which data access is granted. The stored encrypted data may be decrypted before encryption with the access encryption parameters. The stored encrypted data may be encrypted with the access encryption parameters without first decrypting the stored encrypted data.

Secure access to the patient's data may be granted in response to the patient's consent to a data access request, based on providing the access encryption parameters to the user granted access. The user granted access may be the user that requested access. The user granted access may be a user designated for access to the data by the user that requested access. A user with access to the data may be granted consent to share the data with another user. The user sharing the data, and the user to whom data is shared, may be provided with separate and distinct access encryption parameters permitting the patient to selectively control access to the patient's data.

The secure access may be granted to a specific user related to the request. For example, in an exemplary implementation, the access encryption parameters configured for a user granted access may be revoked based on a change in the user's login or registration status. The user may be required to periodically authenticate their identity to maintain access to the patient's data. For example, the user may be required to periodically enter authentication data such as, for example, a password, a predetermined authentication pattern, or a digital security certificate, to maintain access to the patient's data. The user may be required to present biometric authentication data such as a gesture, swipe pattern, face picture, voice print, or fingerprint, to maintain access to the data. The user may be required to continuously present biometric authentication data to maintain authentication. For example, the user may be required to maintain their finger in contact with a sensor configured to capture the user's fingerprint, to maintain access to the patient's data. In an illustrative example, the user's access may be granted while the finger remains in contact with the sensor, and the user's access may be revoked when the finger is removed from the sensor. The user may be prompted by a mobile device application or other software to enter the authentication data. The authentication data entered during a predetermined period of time, in multiple authentication scenarios, may be stored, and accumulated with previously entered authentication data. The accumulated authentication data may be sampled. The sampled accumulated authentication data may be evaluated to validate the authentication data provided by the user. For example, the sampled accumulated authentication data may be evaluated based on techniques as would be available to one of ordinary skill in the arts of statistics, probability, image processing, audio processing, or digital signal processing, to determine if the authentication data may be anomalous. For example, the authentication data may be determined anomalous if the authentication data changes less over time than may be expected in the case of biometric authentication data captured from a live person. In some implementations, the access encryption parameters may be revoked if the user fails to authenticate, or if an anomaly detected in biometric authentication data accumulated over time suggests the biometric authentication data may not be representative of a live person. Examples of changes over time in biometric data that may indicate the data are not representative of a live person may include absence of eye blinking, fixed facial expression, absence of breathing movement, remaining still for extended periods, and the like. Other such indicia of biometric data not representative of a live person as would be known by one of ordinary skill may be employed by various implementations to authenticate the liveness of a person attempting to authenticate their identity to gain access to a patient's medical records.

The secure access may be granted to a specific device related to the request. The access encryption parameters may be revoked based on device identification. The device may be identified by a digital security certificate. The digital security certificate may be encoded by the device. The digital security certificate, or data determined as a function of the digital security certificate, may be presented by the device in response to a request to authenticate the device identity. The digital security certificate may be associated to the device. The device may be identified by wireless or wireline communication interface parameters such as a MAC address, MEID, IMEI, Bluetooth® address, or the like. The device may be identified based on presenting communication interface parameters captured during device registration. Some implementations may maintain a list of devices granted access, based on a device communication interface parameter or a digital security certificate associated to the device. The communication interface parameter identifying a device may be captured by a trusted wireless or wireline device configured to detect the communication interface parameters of the device being identified. In a non-limiting example, a mobile device application operated by a trusted user such as an administrator could be configured to capture and decode the Wi-Fi MAC address transmitted by a device being identified. The trusted device could be configured to relay to a central server the captured communication interface parameters of the device being identified, permitting the central server to identify the device based on comparing a communication interface parameter captured during authentication with a communication interface parameter captured during registration. Some implementations may maintain a list of communication interface parameters corresponding to devices denied access. For example, a trusted device may be configured to periodically scan various network media, to capture communication interface parameters accessible by the trusted device. The trusted device may compare the captured communication interface parameters to the list of devices denied access. In some implementations, the access encryption parameters configured for a trusted device may be temporarily or permanently revoked if a device denied access appears in a periodic scan of active communication interface parameters. For example, access by an authorized device may be revoked for a predetermined period of time in response to the appearance nearby of an unauthorized device's communication interface parameter. The revoked access may be restored to the authorized device in response to determining the unauthorized device has moved away. In an illustrative example, such revocation of trusted device access when an untrusted device is nearby may prevent unauthorized access, such as by eavesdropping from unauthorized devices.

The secure access may be granted at a specific location related to the request. In some implementations, the access encryption parameters may be revoked based on device location. The device location may be determined by the device or a trusted device monitoring the location of the device granted access. The device location may be determined based on GPS configured in the device. The device location may be determined based on the device capturing a communication interface parameter transmitted by a nearby device having a known location, such as, for example, the Wi-Fi MAC address of a trusted wireless access point. The device location may be determined by a trusted device capturing a communication interface parameter transmitted by the device being located. Some implementations may maintain a list of locations from which access is granted. In an illustrative example, access may be configured within a maximum distance from a location where access is granted. In an exemplary implementation, a contingent location access time period may be configured in which access is maintained, when a device leaves a location at which access is granted. In an illustrative example, a doctor may be granted access at their primary practice location, at home, and at a hospital, with a thirty minute contingent location access time period. In this example, the doctor may have continuous access while traveling within the thirty minute time period between the locations where access was granted. In this scenario, the doctor's access may be revoked if the doctor does not arrive within the contingent location access time period at a location where the doctor has been granted access.

The secure access may be configured at a specific time related to the request. The configured secure access may be scheduled to activate at a predetermined time.

The secure access may be granted for a specific period of time related to the request. The access encryption parameters may be revoked when the time period granted to access the data has passed.

The access encryption parameters may be revoked in response to a change in patient consent to access the data. For example, the access encryption parameters may be revoked in response to the patient withdrawing consent to access the data.

Distinct access encryption parameters may be configured in response to each request of multiple requests to access a patient's health record data. The personal health record data may be encrypted with distinct access encryption parameters configured for each access request. Distinct access encryption parameters may be configured for selective access to individual data fields of a personal health record, permitting selective control of access to the individual data fields.

The access encryption parameters may be stored separated from the data to which access is granted.

An implementation may configure secure access to a patient's stored health record data encrypted with storage encryption parameters, based on encrypting the stored data with access encryption parameters distinct from the storage encryption parameters, in response to patient consent to a data access request, and automatically provide the secure access to a specific user device at a time related to the request.

Data may be protected by encryption based on Symmetric Encryption & Key Management (SE&KM), using techniques such as those disclosed with reference to FIGS. 1-14 of U.S. Pat. No. 7,362,868, entitled “Hidden link dynamic key manager for use in computer systems with database structure for storage of encrypted data and method for storage and retrieval of encrypted data,” filed Oct. 20, 2000.

Data may be encrypted with a unique encryption key.

Encrypted stored data may be decrypted to clear-text, and the clear-text then encrypted using a per request session key, suitable for protection-in-transit.

When in-transit protected data arrives from the client to the server-side application, transport encryption may be removed, and then SE&KM may be applied for data-at-rest protection.

Individual encryption keys may be stored separately. Individual encryption keys may be encrypted. Access to each encryption key may be individually controllable. Any user's right to access a particular piece of information may be granted, and subsequently revoked in real-time.

Links between encryption keys and corresponding data items may be encrypted, preventing denial of service attacks through targeted key destructions.

Data copies, even in unknown locations, may be protected through risk transfer (from data to keys). Security is made a property of data and not the underlying infrastructure.

The system may be configured to track all operations (including any access to protected data items) based on logical key centralization.

The system may be configured to implement digital shredding of data items, even in unknown locations, based on verifiable destruction of corresponding encryption keys.

The system may be configured to protect relationships between data items, to permit de-identification and anonymization.

The details of various aspects are set forth in the accompanying drawings and the description below. Other features and advantages will be apparent from the description and drawings, and from the claims.

### BRIEF DESCRIPTION OF THE DRAWINGS

FIG. 1 depicts an exemplary operational scenario configuring secure access to a patient's stored health record data, in response to receiving the patient's consent to access the data.

FIG. 2 depicts a schematic view of an exemplary consent controlled PHR (Personal Health Record) access network configured to provide secure access to a patient's stored health record data, in response to receiving the patient's consent to access the data.

FIG. 3 depicts a structural view of an exemplary consent controlled PHR access device configured with a PHRAE (Personal Health Record Access Engine) to provide secure access to a patient's stored health record data, in response to receiving the patient's consent to access the data.

FIG. 4 depicts an exemplary PHR sharing scenario based on an exemplary consent controlled PHR access implementation.

FIG. 5 depicts an exemplary PHR process flow based on an exemplary consent controlled PHR access implementation.

FIG. 6 depicts an exemplary consent controlled PHR data security implementation.

FIG. 7 depicts an exemplary PHR data flow based on an exemplary consent controlled PHR access implementation.

FIG. 8 depicts a process flow of an exemplary PHRAE platform aspect implementation configured to provide secure access to a patient's stored health record data, in response to receiving the patient's consent to access the data.

FIG. 9 depicts a process flow of an exemplary PHRAE patient aspect implementation configured to provide secure access to a patient's stored health record data, in response to receiving the patient's consent to access the data.

FIG. 10 depicts a process flow of an exemplary PHRAE provider aspect implementation configured to provide secure access to a patient's stored health record data, in response to receiving the patient's consent to access the data.

FIG. 11 depicts a process flow of an exemplary PHRAE system aspect configured to provide secure access to a patient's stored health record data, in response to receiving the patient's consent to access the data.

Like reference symbols in the various drawings indicate like elements.

### DETAILED DESCRIPTION

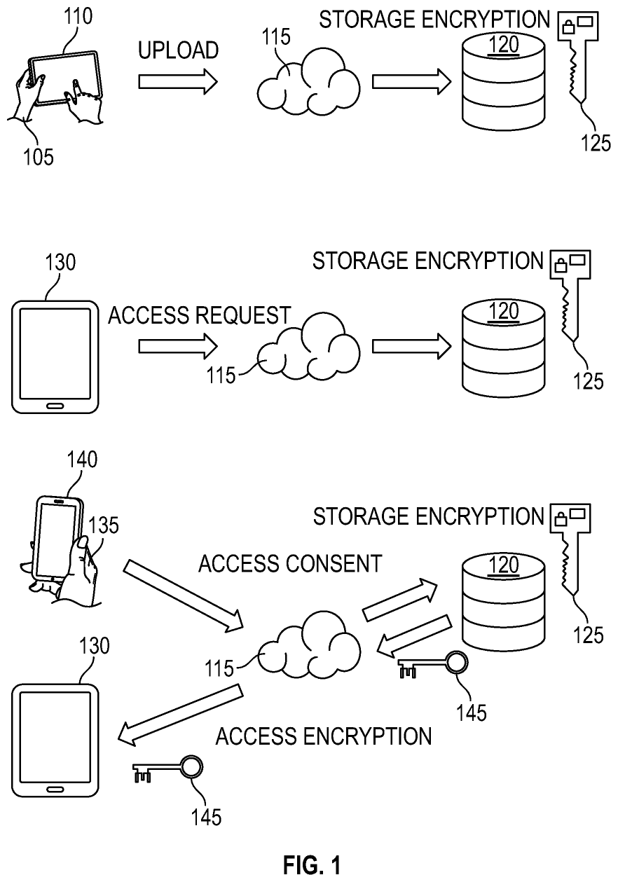

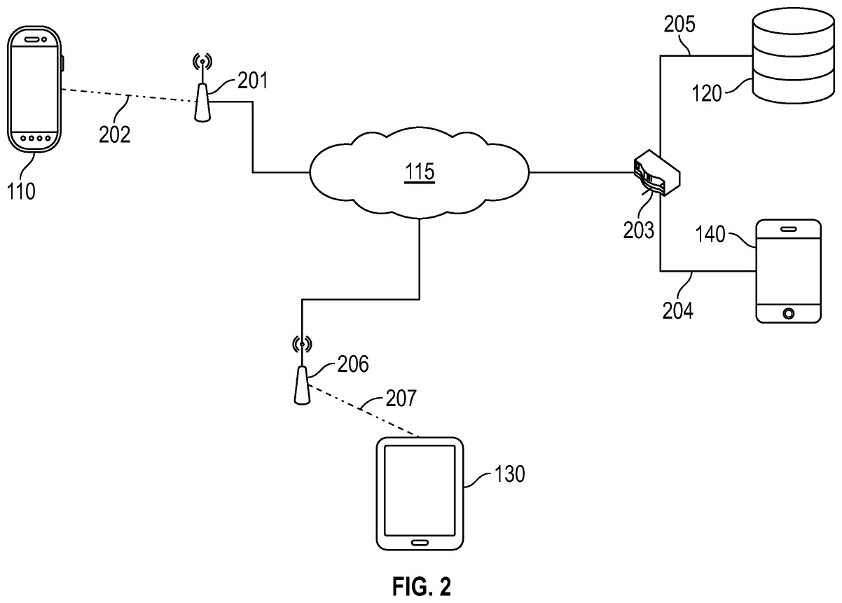

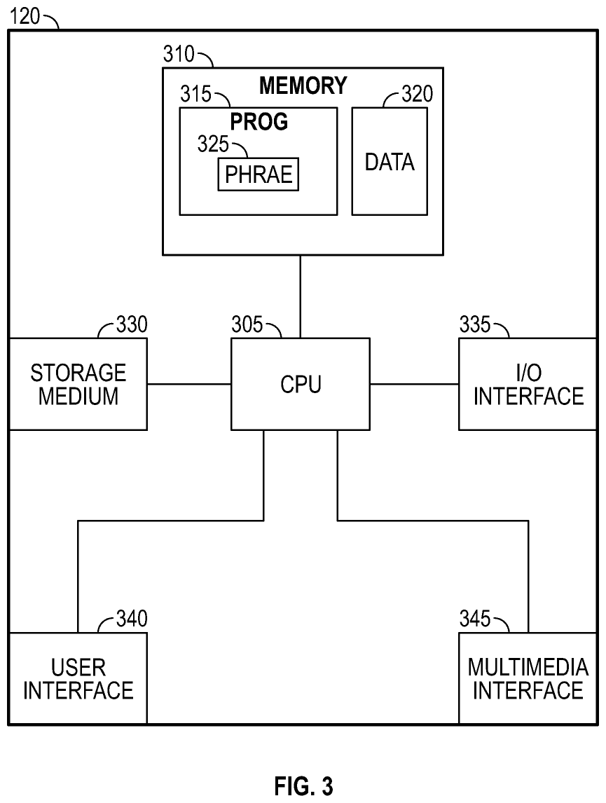

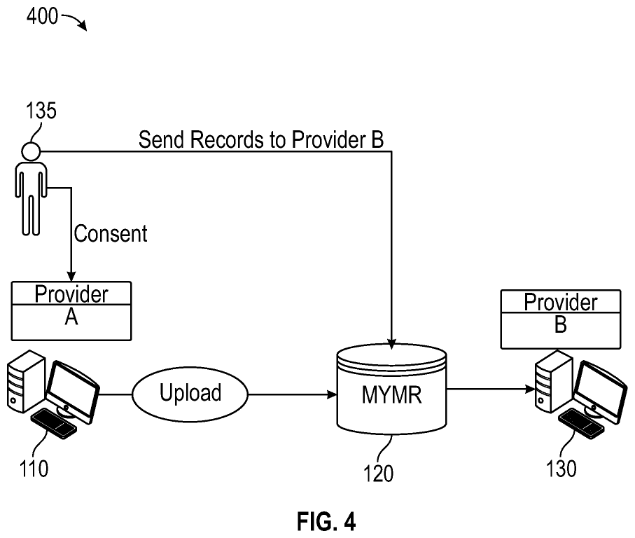

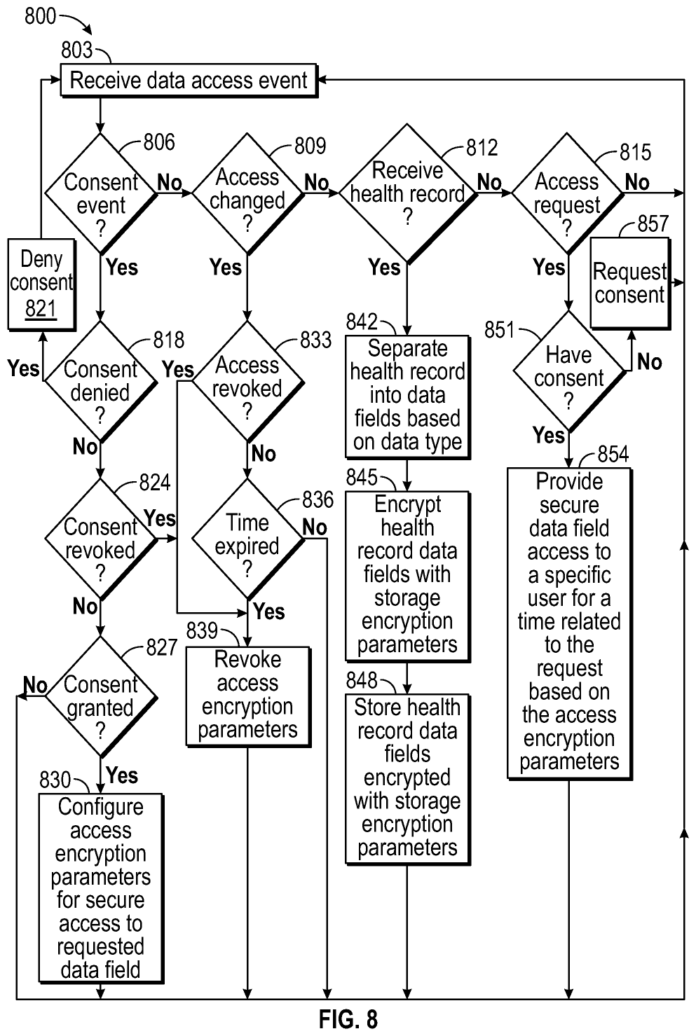

To aid understanding, this document is organized as follows. First, configuring secure access to a patient's stored health record data, in response to receiving the patient's consent to access the data, is briefly introduced with reference to FIG. 1. Second, with reference to FIGS. 2-3, the discussion turns to exemplary implementations that illustrate consent controlled PHR (Personal Health Record) access system design. Specifically, exemplary consent controlled PHR access network and computing device implementations are disclosed. Then, with reference to FIGS. 4-7, illustrative consent controlled PHR access scenarios are presented to explain improvements in PHR technology. Finally, with reference to FIGS. 8-11, exemplary process flows depicting various aspects of consent controlled PHR access system implementation are described.

FIG. 1 depicts an exemplary operational scenario configuring secure access to a patient's stored health record data, in response to receiving the patient's consent to access the data. In FIG. 1, the provider 105 operates the provider A device 110 operably coupled via the network cloud 115 with the PHR platform 120. The provider 105 uploads a patient's personal health record data to the PHR platform 120. In the depicted example, the provider 105 is a doctor. In the illustrated example, the personal health record data is medical data generated as a result of the doctor treating the patient. The PHR platform 120 stores the uploaded personal health record data in the patient's medical record storage account secured by the PHR platform 120. The PHR platform 120 secures the patient health record data encrypted at rest based on storage encryption parameters 125.

In the depicted example, the PHR platform 120 receives a request from the provider B device 130 for access to the patient's personal health record data. The access request received from the provider B device 130 may be initiated by a provider such as an insurer, medical doctor, or hospital requiring access to the patient's personal health record data. In the depicted example, the PHR platform 120 determines the provider B device 130 does not have permission to access the requested data.

In the depicted implementation example, the PHR platform 120 notifies the patient 135 that a provider has requested access to the patient's personal health record data. In the depicted example, the patient 135 receives the notification via the patient device 140. In the illustrated example, the patient device 140 presents the patient 135 with details of the request to access the patient's personal health record data. In the depicted example, the patient 135 reviews the details of the request to access the patient's personal health record data using the patient device 140. The patient 135 decides the provider requesting access to the patient's personal health record data should be permitted to access the patient's data. The patient 135 gives consent for the provider B device 130 to access the patient's personal health record data that was uploaded by the provider 105. In response to receiving the patient's consent to the personal health record data access request, the PHR platform 120 configures secure access for the requestor to access the patient's stored personal health record data. In the illustrated example, the PHR platform 120 configures the secure access for the requestor based on securing the data with access encryption parameters 145 configured for the requestor. The access encryption parameters 145 are distinct from the storage encryption parameters 125. The access encryption parameters 145 may include a unique session key valid only for the duration of access granted by the patient 135. The provider B device 130 may use the access encryption parameters 145 to securely access the patient's personal health record data. The patient 135 may withdraw consent at any time to revoke the access encryption parameters 145 configured for the requestor, thereby denying the requestor further access to the patient's data.

FIG. 2 depicts a schematic view of an exemplary consent controlled PHR access network configured to provide secure access to a patient's stored health record data, in response to receiving the patient's consent to access the data. In FIG. 2, according to an exemplary implementation, data may be transferred to the system, stored by the system and/or transferred by the system to users of the system across local area networks (LANs) or wide area networks (WANs). In accordance with various implementations, the system may include numerous servers, data mining hardware, computing devices, or any combination thereof, communicatively connected across one or more LANs and/or WANs. One of ordinary skill in the art would appreciate that there are numerous manners in which the system could be configured, and implementations of the present disclosure are contemplated for use with any configuration. Referring to FIG. 2, a schematic overview of a system implementation in accordance with the present disclosure is shown. In the depicted implementation, the exemplary system includes the exemplary provider A device 110. In the depicted implementation example, the provider A device 110 is a mobile computing device hosting a mobile app configured to present a provider with a user interface permitting the provider to request access, upload, download, share, and manage patient personal health record data. In the illustrated implementation, the PHR platform 120 is a cloud database configured to securely store and provide access to patient personal health record data in response to the patient providing consent for access to the data. In the depicted implementation, the provider B device 130 is a mobile computing device hosting a mobile app configured to present a provider with a user interface permitting the provider to request access, upload, download, share, and manage patient personal health record data. In the depicted implementation, the patient device 140 is a mobile computing device hosting a mobile app configured to provide a user interface permitting a patient to assert ownership control over the patient's personal health record data secured by the PHR platform 120. In the illustrated implementation, the provider A device 110 is communicatively and operably coupled by the wireless access point 201 and the wireless link 202 with the network cloud 115 (for example, the Internet) to send, retrieve, or manipulate information in storage devices, servers, and network components, and exchange information with various other systems and devices via the network cloud 115. In the depicted implementation, the illustrative system includes the router 203 configured to couple the patient device 140 communicatively and operably to the network cloud 115 via the communication link 204. In the illustrated example, the router 203 also communicatively and operably couples the PHR platform 120 to the network cloud 115 via the communication link 205. In the depicted implementation, the provider B device 130 is communicatively and operably coupled with the network cloud 115 by the wireless access point 206 and the wireless communication link 207. In various implementations, one or more of: the provider A device 110, the PHR platform 120, the provider B device 130, or the patient device 140 may include an application server configured to store or provide access to information used by the system. In some implementations, one or more application server may retrieve or manipulate information in storage devices and exchange information through the network cloud 115. In various implementations, one or more of: the provider A device 110, the PHR platform 120, the provider B device 130, or the patient device 140 may include various applications implemented as processor-executable program instructions. Various processor-executable program instruction applications may also be configured in some implementations, to manipulate information stored remotely and process and analyze data stored remotely across the network cloud 115 (for example, the Internet). According to an exemplary implementation, as shown in FIG. 2, exchange of information through the network cloud 115 or other network may occur through one or more high speed connections. In some cases, high speed connections may be over-the-air (OTA), passed through networked systems, directly connected to one or more network cloud 115 or directed through one or more router. In various implementations, one or more router may be optional, and other implementations in accordance with the present disclosure may or may not utilize one or more router. One of ordinary skill in the art would appreciate that there are numerous ways any or all of the depicted devices may connect with the network cloud 115 for the exchange of information, and implementations of the present disclosure are contemplated for use with any method for connecting to networks for the purpose of exchanging information. Further, while this application may refer to high speed connections, implementations of the present disclosure may be utilized with connections of any useful speed. In an implementation example, components or modules of the system may connect to one or more of: the provider A device 110, the PHR platform 120, the provider B device 130, or the patient device 140 via the network cloud 115 or other network in numerous ways. For instance, a component or module may connect to the system i) through a computing device directly connected to the network cloud 115, ii) through a computing device connected to the network cloud 115 through a routing device, or iii) through a computing device connected to a wireless access point. One of ordinary skill in the art will appreciate that there are numerous ways that a component or module may connect to a device via network cloud 115 or other network, and implementations of the present disclosure are contemplated for use with any network connection method. In various examples, one or more of: the provider A device 110, the PHR platform 120, the provider B device 130, or the patient device 140 could include a personal computing device, such as a smartphone, tablet computer, wearable computing device, cloud-based computing device, virtual computing device, or desktop computing device, configured to operate as a host for other computing devices to connect to. One or more communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with implementations of the present disclosure, and implementations of the present disclosure are contemplated for use with any communications means.

FIG. 3 depicts a structural view of an exemplary consent controlled PHR access device configured with a PHRAE (Personal Health Record Access Engine) to provide secure access to a patient's stored health record data, in response to receiving the patient's consent to access the data. The exemplary consent controlled PHR access device depicted in FIG. 3 is the PHR platform 120 (also depicted at least in FIG. 1 and FIG. 2). The implementation disclosed with reference to FIG. 3 may also be understood by one of ordinary skill as representative of exemplary implementations of provider A device 110, provider B device 130, or patient device 140 (also depicted at least in FIG. 1 and FIG. 2).

In FIG. 3, the block diagram of the exemplary PHR platform 120 includes the processor 305 and the memory 310. In FIG. 3, the processor 305 is in electrical communication with the memory 310. The depicted memory 310 includes the program memory 315 and the data memory 320. The depicted program memory 315 includes processor-executable program instructions implementing the PHRAE (Personal Health Record Access Engine) 325. The illustrated program memory 315 may encode processor-executable program instructions configured to implement an OS (Operating System). The OS may include processor executable program instructions configured to implement various operations when executed by the processor 305. The OS may be omitted. The illustrated program memory 315 may encode processor-executable program instructions configured to implement various Application Software. The Application Software may include processor executable program instructions configured to implement various operations when executed by the processor 305 The Application Software may include one or more database application. The Application Software may be omitted. In the depicted implementation, the processor 305 is communicatively and operably coupled with the storage medium 330. The storage medium 330 may include cloud data storage accessible to the processor 305. In the depicted implementation, the processor 305 is communicatively and operably coupled with the I/O (Input/Output) interface 335. In the depicted implementation, the I/O interface 335 includes a network interface. The network interface may be a wireless network interface. The network interface may be a Wi-Fi interface. The network interface may be a Bluetooth® interface. The PHR platform 120 may include more than one network interface. The network interface may be a wireline interface. The network interface may be omitted. The consent controlled PHR access device depicted in FIG. 3 may include a GPS (Global Positioning System) module operably coupled with the processor 305 to permit the processor 305 to determine the location of the device. In the depicted implementation, the processor 305 is communicatively and operably coupled with the user interface 340. In the depicted implementation, the processor 305 is communicatively and operably coupled with the multimedia interface 345. In the illustrated implementation, the multimedia interface 345 includes interfaces adapted to input and output of audio, video, and image data. The multimedia interface 345 may include one or more still image camera or video camera. Useful examples of the illustrated PHR platform 120 include, but are not limited to, personal computers, servers, tablet PCs, smartphones, or other computing devices. Multiple PHR platform 120 devices may be operably linked to form a computer network in a manner as to distribute and share one or more resources, such as clustered computing devices and server banks/farms. Various arrangements of such general-purpose multi-unit computer networks suitable for implementations of the disclosure, their typical configuration, and standardized communication links are well known to one skilled in the art, as explained in more detail in the foregoing FIG. 2 description. An exemplary PHR platform 120 design may be realized in a distributed implementation. Some PHR platform 120 designs may be partitioned between a client device, such as, for example, a phone, and a more powerful server system, as depicted, for example, in FIG. 2. A PHR platform 120 partition hosted on a PC or mobile device may choose to delegate some parts of computation, such as, for example, machine learning or deep learning, to a host server. A client device partition may delegate computation-intensive tasks to a host server to take advantage of a more powerful processor, or to offload excess work. Some PHR platform 120 devices may be configured with a mobile chip including an engine adapted to implement specialized processing, such as, for example, neural networks, machine learning, artificial intelligence, image recognition, audio processing, or digital signal processing. Such an engine adapted to specialized processing may have sufficient processing power to implement some PHR platform 120 features. However, an exemplary PHR platform 120 may be configured to operate on a device with less processing power, such as, for example, various gaming consoles, which may not have sufficient processor power, or a suitable CPU architecture, to adequately support PHR platform 120. Various implementations configured to operate on a such a device with reduced processor power may work in conjunction with a more powerful server system.

FIG. 4 depicts an exemplary PHR sharing scenario based on an exemplary consent controlled PHR access implementation. In FIG. 4, the exemplary PHR sharing scenario 400 includes the patient 135 giving consent to the Provider A using the provider A device 110 to upload the patient's medical records into the patient's medical record storage account secured by the PHR platform 120. In the depicted example, the patient 135 may then give consent to the Provider A to share the patient's medical records with Provider B to access the patient's medical records using the Provider B device 130. The medical record access granted to Provider A and Provider B in response to the access consent given by the patient 135 is configured by the PHR platform 120. The patient 135 may grant or deny access to all or part of their medical records. For example, secure access to various data fields within the patient 135 medical records may be individually configured for each requestor and for each field. The patient 135 may, for example, grant Provider A access to all of the patient 135 medical records secured by the PHR platform 120. The patient 135 may, for example, grant Provider A consent to share only data from medical laboratory test results related to a particular condition, while denying permission share the remainder of the patient 135 medical records. The patient 135 may consent to access by Provider B to the specific medical record data fields shared from Provider A to Provider B. The patient 135 controls their medical records using the PHR platform 120, permitting secure sharing with any provider or doctor using a PHR platform 120 interface application, in response to patient consent to access the patient's data. The patient 135 may control the duration of the access that the providers may be granted, and the access may be revoked at any time based on the patient 135 withdrawing consent to access the patient 135 medical records. The patient 135 use of the PHR platform 120 to control access to the patient's medical records may permit the patient 135 to avoid having to either give written consent to send the generated medical records to another provider for future treatment, or travel with a digital or paper copy of the records.

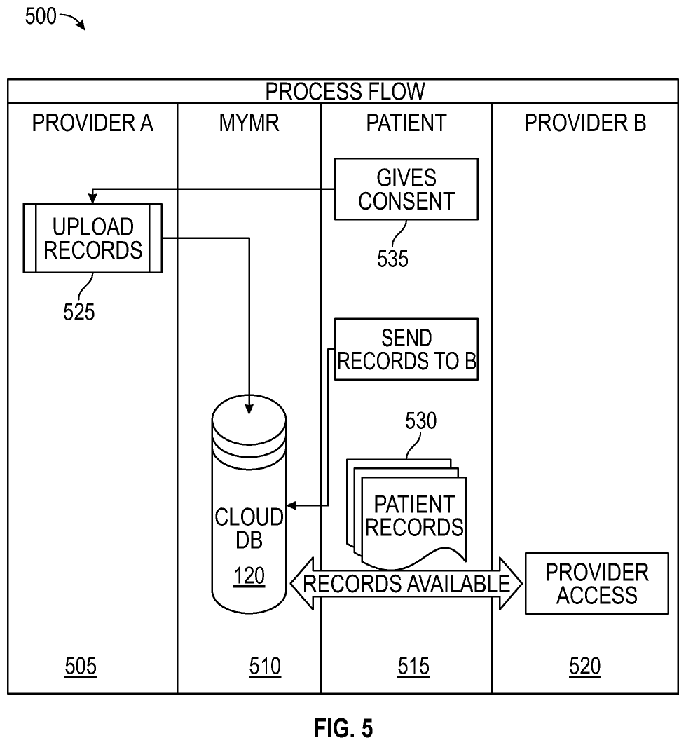

FIG. 5 depicts an exemplary PHR process flow based on an exemplary consent controlled PHR access implementation. In FIG. 5, the exemplary PHR process flow 500 includes the steps provider A device upload 505, PHR platform device store 510, patient device consent 515, and provider B device access 520.

The exemplary process begins with the step provider A device upload 505, with provider A generating medical data related to a patient. The patient authorizes the provider A device upload 525 of the patient's medical data to the patient's medical record storage secured by the PHR platform 120. The PHR platform 120 may be referred to as MyMR. In the depicted example, the PHR platform 120 includes a cloud database configured to store the patient's medical record data uploaded by provider A. In the PHR platform device store 510 step, the PHR platform 120 stores the patient's medical record data uploaded by provider A. In the patient device consent 515 step, the patient authorizes the PHR platform 120 to send the patient records 530 to provider B in response to the PHR platform 120 receiving the patient share consent 535. In the provider B device access 520 step, provider B accesses the patient records 530 made available by the PHR platform 120.

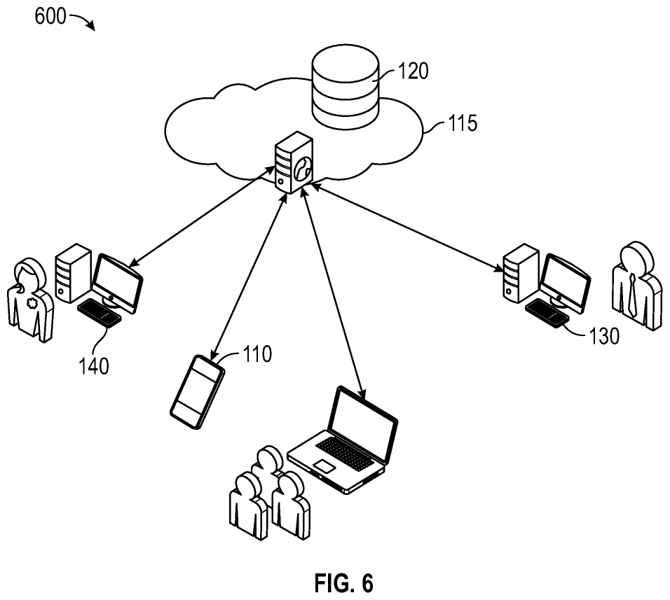

FIG. 6 depicts an exemplary consent controlled PHR data security implementation. In FIG. 6, the exemplary PHR data security implementation 600 includes the provider A device 110, the provider B device 130, and the patient device 140 communicatively coupled with the PHR platform 120 via the network cloud 115. In the depicted example, the provider A device 110, the provider B device 130, and the patient device 140 host an interface application configured to permit a patient to control provider access to the patient's medical records using the PHR platform 120. The interface application may be a smartphone, desktop, or tablet application. Additional providers using other devices configured with a PHR platform 120 interface application may also request and obtain access to the patient's medical records in response to consent given by the patient. In the depicted example, the communication links coupling the PHR platform 120 with the provider A device 110, the provider B device 130, and the patient device 140 secure the patient medical record data access using SE&KM technology. In an illustrative example, the mechanism of SE&KM allows secure transfer of data over the internet. All data transferred by the provider A device 110, the PHR platform 120, the provider B device 130, and the patient device 140 are secured using SE&KM technology providing the highest level of encryption and therefore protect the sensitive medical data. In an illustrative example, the following explains some features of an exemplary SE&KM mechanism: 1) Every logical data item is encrypted with a unique key, thus limiting the exposure surface of any possible leak; 2) Keys are stored separately, encrypted, and access to every key is individually controllable; Anyone's right to access a particular piece of information can be granted, and subsequently revoked in real-time; 3) Links between keys and corresponding data items are encrypted, preventing denial of service attacks through targeted key destructions; 4) Data copies, even in unknown locations, are protected through risk transfer (from data to keys); Security is made a property of data and not the underlying infrastructure; 5) Due to logical key centralization, all operations performed by the system (including any access to protected data items) may be tracked very granularly; 6) “Digital shredding” of data items, even in unknown locations, is possible through verifiable destruction of corresponding encryption keys; and, 7) Relationships between data items are protected, as well, providing for de-identification and anonymization.

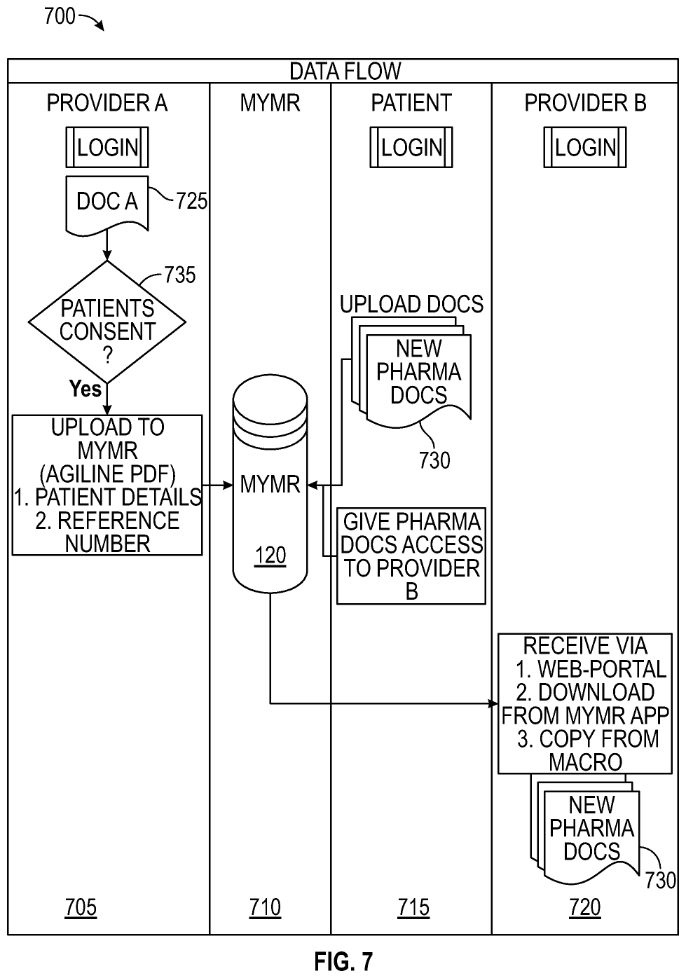

FIG. 7 depicts an exemplary PHR data flow based on an exemplary consent controlled PHR access implementation. In FIG. 7, the exemplary PHR data flow 700 includes the provider A device data flow 705, the PHR platform device data flow 710, the patient device data flow 715, and the provider B device data flow 720.

In the depicted implementation, the provider A device data flow 705 includes provider A generating the medical record document 725 and logging in to the PHR platform 120 to request patient authorization for uploading the medical record document 725. The PHR platform 120 may be referred to as MyMR. Provider A sends an upload consent request to the PHR platform 120, to request patient authorization to upload the medical record document 725 to the patient's medical record storage secured by the PHR platform 120. In the depicted example, provider A receives a notification that secure access has been configured for provider A to upload the medical record 725 to the patient's medical record storage secured by the PHR platform 120 in response to the patient consent 735, and provider A uploads the medical record document 725.

In the illustrated example, the PHR platform device data flow 710 includes processing the provider A login, and configuring secure access for provider A to upload the medical record document 725 generated by provider A, in response to receiving consent from the patient for provider A to upload to the patient's medical record storage secured by the PHR platform 120. In the depicted example, the PHR platform device data flow 710 also includes processing the patient login and the provider B login. In an illustrative implementation example, the PHR platform 120 (MyMR) is configured to be adaptable to work with any EHR (Electronic Health Record) format.

For example, patient medical records may be generated by providers and initially stored in the provider's EHR systems. A MyMR implementation may facilitate transfer of records from various dissimilar provider systems in diverse record formats using a proprietary PDF File Generator application that pushes medical record documents into a MyMR system for ingest. In an illustrative example, a MyMR desktop application may access the PDF File Generator to access the medical record data rendered by the PDF File Generator.

In the depicted example, the PHR platform 120 receives the medical record document 725 uploaded by provider A, visually renders the document via a PDF File Generator, processes the rendered document with an extraction utility to extract data from the document, splits the data into individual data fields, and securely stores the data fields. In the depicted example, data is extracted from the document using a data extraction utility, for example, a PDF File Generator. In the illustrated example, the data extracted from the medical record document 725 are uploaded into a MyMR web application, where the recipient details and reference number are added, and then transferred to the patient's medical record storage secured by the PHR platform 120.

A MyMR implementation may use AI (Artificial Intelligence) technology to split a medical record into subsections, and store the subsections in respective folders. The medical record subsections may be data fields extracted from the medical record. The AI technology may identify the data type of a medical record data field. The AI technology may categorize the medical record data field based on the data type. For example, a medical record may include physician notes, allergy information, drug history, radiology information, and the like. The AI technology may be configured to split medical record document pages according to the header, identify data fields in the medical record, extract data encoded by the identified data fields, determine the data type of the data extracted from the data fields, store the data extracted from the medical record data fields in respective folders selected based on the data type, and provide access to the data in the data fields.

In the illustrated implementation, the patient device data flow 715 includes the patient logging in to the PHR platform 120. After logging in, the patient receives a notification from the PHR platform 120 that provider A has requested authorization to upload the medical record document 725 to the patient's medical record storage secured by the PHR platform 120. The patient gives consent for provider A to upload the medical record document 725, and the PHR platform 120 provides access to provider A to upload the medical record 725. The patient generates the pharmacy documents 730 and the patient uploads the pharmacy documents 730 to the patient's medical record storage secured by the PHR platform 120. The patient gives consent through the PHR platform 120 for provider B to access to the patient's pharmacy documents 730.

In the depicted example, the provider B device data flow 720 includes provider B logging in to the PHR platform 120. In the illustrated example, provider B receives a notification that secure access has been configured for provider B to access the pharmacy documents 730 uploaded by the patient, in response to the patient's consent. In the depicted implementation, provider B may access the pharmacy documents 730 by at least three methods, including: 1) Download the documents from the PHR platform 120 web portal; 2) Download the documents from the MyMR app; or, 3) Copy the documents from a macro.

In an illustrative MyMR implementation example, a macro is a program configured to interact with an EHR to search and add patient information. The macro may copy the necessary information from the fields in a document using an extraction utility. For example, the document may be a PDF document. The extraction utility may be a PDF extraction utility, such as, for example, a PDF File Generator. The exemplary MyMR implementation may input the information extracted from a document field to an EHR form configured to ingest the information into the MyMR implementation. In an illustrative example, various MyMR implementations may support GMed, GE Centricity, and Allscripts, and may provide macros to input data extracted from various document types into other EHR types. A MyMR implementation may provide a user interface configured to permit a user to customize a macro to interact with an EHR and document format that is not supported by an existing macro. Such macro customization may permit a user to achieve efficient ingest of medical record data from EHRs configured in a format previously unsupported for automatic EHR data ingest. An exemplary MyMR macro implementation may encourage interaction with multiple EHR platforms in the future.

FIG. 8 depicts a process flow of an exemplary PHRAE platform aspect implementation configured to provide secure access to a patient's stored health record data, in response to receiving the patient's consent to access the data. In FIG. 8, the depicted method is given from the perspective of the Personal Health Record Access Engine (PHRAE) 325 implemented via processor-executable program instructions executing on the PHR platform 120 processor 305, depicted in FIG. 3. In various implementations, the method depicted in FIG. 8 may also be understood by one of ordinary skill as if given from the perspective of the provider A device 110, the provider B device 130, or the patient device 140, implemented via processor-executable program instructions executing on a processor configured in the respective device. The provider A device 110, the provider B device 130, and the patient device 140 are depicted at least in FIG. 1 and FIG. 2. In the illustrated implementation, the PHRAE 325 executes as program instructions on the processor 305 configured in the PHRAE 325 host PHR platform 120, depicted in at least FIG. 1, FIG. 2, and FIG. 3. In some implementations, the PHRAE 325 may execute as a cloud service communicatively and operatively coupled with system services, hardware resources, or software elements local to and/or external to the PHRAE 325 host PHR platform 120.

In an illustrative example, the PHRAE 325 implementation executing as program instructions on the processor 305 may be designed with event-driven or interrupt-driven embedded programming techniques to detect and process events based on received messages, captured sensor data, user input, or other event triggers as would be known to one of ordinary skill in the art of embedded control systems. Some PHRAE 325 implementations may designate various events as having different priorities, as a design decision within the scope of one having ordinary skill in the art. Various exemplary PHRAE 325 implementations may process events having different priorities in an order different from the order in which the events were detected or different from the order in which the events occurred, based on configured event priority determined by a designer having ordinary skill in the art. In an illustrative example, the exemplary PHRAE 325 process flow given with reference to the drawing represents an example of real-time asynchronous event handling, and an exemplary PHRAE 325 process implementation may handle events in a different order as a result of real-time conditions, design decisions, and other factors, as the skilled artisan would recognize. For example, an exemplary PHRAE 325 process may prioritize an event related to user privacy or data security for handling before an event related to file system maintenance.

The depicted method 800 begins at step 803 with the processor 305 receiving a data access event. The data access event received by the processor 305 may include an event type. The received data access event may result from a change in patient consent, a data access authorization change, a received health record, an access request, or other activity.

Then, the method continues at step 806 with the processor 305 performing a test to determine if the data access event received by the processor 305 is a consent event. Upon a determination by the processor 305 at step 806 the data access event is not a consent event, the method continues at step 809. Upon a determination by the processor 305 at step 806 the data access event is a consent event, the method continues at step 818.

At step 809 the processor 305 performs a test to determine if the data access event received by the processor 305 is a change in access. Upon a determination by the processor 305 at step 809 the data access event is not a change in access, the method continues at step 812. Upon a determination by the processor 305 at step 809 the data access event is a change in access, the method continues at step 833.

At step 812, the processor 305 performs a test to determine if the data access event received by the processor 305 at step 803 is a received health record. Upon a determination by the processor 305 at step 812 the data access event received by the processor 305 at step 803 is not a received health record, the method continues at step 815. Upon a determination by the processor 305 at step 812 the data access event received by the processor 305 at step 803 is a received health record, the method continues at step 842.

At step 815, the processor 305 performs a test to determine if the data access event received by the processor 305 at step 803 is an access request. Upon a determination by the processor 305 at step 815 the data access event received by the processor 305 at step 803 is not an access request, the method continues at step 803. Upon a determination by the processor 305 at step 815 the data access event received by the processor 305 at step 803 is an access request, the method continues at step 851.

At step 818, the processor 305 performs a test to determine if the consent event detected by the processor 305 at step 806 should be handled as a consent denied event. For example, the processor 305 may determine the consent event should be handled as a consent denied event in response to a data access attempt by an unauthorized process, or in response to a patient denying access to a provider. Upon a determination by the processor 305 at step 818 the consent event detected by the processor 305 at step 806 should be handled as a consent denied event, the method continues at step 821 with the processor 305 denying consent. The processor 305 at step 821 may deny consent based on sending a digital message to a requestor device, updating state, or doing nothing. In any case, the method continues at step 803. Upon a determination by the processor 305 at step 818 the consent event detected by the processor 305 at step 806 should not be handled as a consent denied event, the method continues at step 824.

At step 824 the processor 305 performs a test to determine if the consent event detected by the processor 305 at step 806 should be handled as a consent revoked event. For example, the processor 305 may determine the consent event should be handled as a consent revoked event in response to a patient withdrawing consent for a provider to access the patient's data. Upon a determination by the processor 305 at step 824 the consent event detected by the processor 305 at step 806 should be handled as a consent revoked event, the method continues at step 839. Upon a determination by the processor 305 at step 824 the consent event detected by the processor 305 at step 806 should not be handled as a consent revoked event, the method continues at step 827.

At step 827, the processor 305 performs a test to determine if the consent event detected by the processor 305 at step 806 should be handled as a consent granted event. For example, the processor 305 may determine the consent event should be handled as a consent granted event in response to a patient giving consent for a provider to access the patient's data. Upon a determination by the processor 305 at step 827 the consent event detected by the processor 305 at step 806 should not be handled as a consent granted event, the method continues at step 803. Upon a determination by the processor 305 at step 827 the consent event detected by the processor 305 at step 806 should be handled as a consent granted event, the method continues at step 830 with the processor 305 configuring access encryption parameters for secure access to the requested data field, and the method continues at step 803.

At step 833, the processor 305 performs a test to determine if the change in access event detected by the processor 305 at step 809 is an access revoked event. The processor 305 may determine the change in access event is an access revoked event in response to a patient revoking a provider's access to the patient's data; due to a change in the provider's authentication status; due to a change in the provider's location; or, due to other factors. Upon a determination by the processor 305 at step 833 the change in access event detected by the processor 305 at step 809 is not an access revoked event, the method continues at step 836. Upon a determination by the processor 305 at step 833 the change in access event detected by the processor 305 at step 809 is an access revoked event, the method continues at step 839.

At step 836, the processor 305 performs a test to determine if the period of time granted to the provider for access to the patient's data has expired. The processor 305 may determine the access time expired based on comparing the current time to an expiration time, or the processor 305 may compare the access time elapsed to the configured access time. Upon a determination by the processor 305 at step 836 the period of time granted to the provider for access to the patient's data has not expired, the method continues at step 803. Upon a determination by the processor 305 at step 836 the period of time granted to the provider for access to the patient's data expired, the method continues at step 839.

At step 839, the processor 305 revokes the access encryption parameters configured by the processor 305 at step 830, and the method continues at step 803.

At step 842, the processor 305 begins the PHR platform ingest of the received health record identified by the processor 305 at step 812 based on processing the received health record data to separate the received health record into data fields based on the data type, and the method continues at step 845. The received health record may be visually rendered by a PDF File Generator process. The received health record may be processed by a data extraction process to harvest data from the rendered health record. The received health record data may be separated into data fields based on processing the health record data with AI technology configured to identify data as a function of the data type. The processor 305 at step 842 may process the health record data using a macro configured to search and add patient information, and copy necessary information from the data fields extracted from the received health record. The method continues at step 845.

At step 845, the processor 305 encrypts the health record data fields with storage encryption parameters. The processor 305 may encrypt each data field with unique storage encryption parameters. The method continues at step 848.

At step 848, the processor 305 stores the health record data fields encrypted with the storage encryption parameters, and the method continues at step 803.

At step 851, the processor 305 performs a test to determine if the requestor has patient consent to access the patient's data by the access request identified by the processor 305 at step 815. The processor 305 may determine if the requestor has patient consent based on comparing the requestor identity, location, request time, device, authentication, or other condition related to the request, with an access condition predetermined as a function of the patient's consent previously granted. Upon a determination by the processor 305 at step 851 the requestor has patient consent to access the patient's data, the method continues at step 854. Upon a determination by the processor 305 at step 851 the requestor does not have patient consent to access the patient's data, the method continues at step 857.

At step 854, the processor 305 configures the PHR platform to provide secure data field access to a specific user for a time related to the request based on the access encryption parameters configured by the processor 305 at step 830. The method continues at step 803.

At step 857, the processor 305 requests consent to access the patient's data. The processor 305 at step 857 may request consent based on sending a digital message to a patient device, updating state, or doing nothing. In any case, the method continues at step 803.

In some implementations, the method may repeat. In various implementations, the method may end.

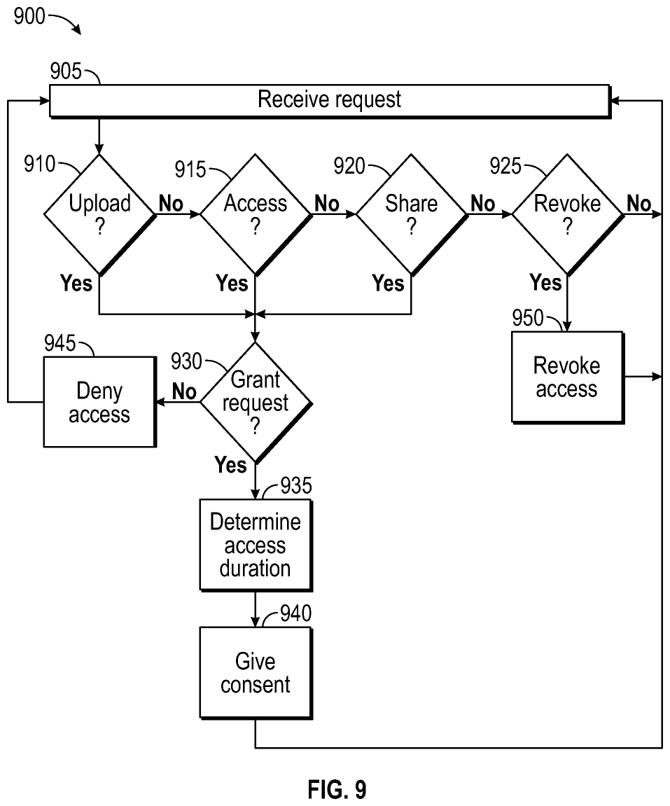

FIG. 9 depicts a process flow of an exemplary PHRAE patient aspect implementation configured to provide secure access to a patient's stored health record data, in response to receiving the patient's consent to access the data. In FIG. 9, the depicted method is given from the perspective of the Personal Health Record Access Engine (PHRAE) 325 implemented via processor-executable program instructions executing on a processor configured in the patient device 140, depicted at least in FIG. 1 and FIG. 2. To facilitate efficient disclosure, the processor 305, depicted in FIG. 3, is identified for this example as representative of the patient device 140 processor. In the illustrated implementation example and using FIG. 3 as representative of the patient device 140, the PHRAE 325 executes as program instructions on the processor 305 configured in the PHRAE 325 host patient device 140, depicted in at least FIG. 1 and FIG. 2. In some implementations, the PHRAE 325 may execute as a cloud service communicatively and operatively coupled with system services, hardware resources, or software elements local to and/or external to the PHRAE 325 host patient device 140.

In an illustrative example, the PHRAE 325 implementation executing as program instructions on the processor 305 may be designed with event-driven or interrupt-driven embedded programming techniques to detect and process events based on received messages, captured sensor data, user input, or other event triggers as would be known to one of ordinary skill in the arts of embedded control systems. Some PHRAE 325 implementations may designate various events as having different priorities, as a design decision within the scope of one having ordinary skill in the art. Various exemplary PHRAE 325 implementations may process events having different priorities in an order different from the order in which the events were detected or different from the order in which the events occurred, based on configured event priority determined by a designer having ordinary skill in the art. In an illustrative example, the exemplary PHRAE 325 process flow given with reference to the drawing represents an example of real-time asynchronous event handling, and an exemplary PHRAE 325 process implementation may handle events in a different order as a result of real-time conditions, design decisions, and other factors, as the skilled artisan would recognize. For example, an exemplary PHRAE 325 process may prioritize an event related to user privacy or data security for handling before an event related to file system maintenance.

The depicted method 900 begins at step 905 with the processor 305 receiving a request. The request may be a request for access to the patient's medical data. The request received by the processor 305 at step 905 may include a request for the patient to provide consent for the requestor to access the medical data controlled by the patient, using the patient's health record storage account secured by the PHR platform 120, depicted at least in FIG. 1, FIG. 2, and FIG. 3. The method continues at step 910.

At step 910 the processor 305 performs a test to determine if the request received by the processor 305 at step 905 is an upload request. Upon a determination by the processor 305 at step 910 the request received by the processor 305 at step 905 is not an upload request, the method continues at step 915. Otherwise, the method continues at step 930.

At step 915 the processor 305 performs a test to determine if the request received by the processor 305 at step 905 is an access request. Upon a determination by the processor 305 at step 915 the request received by the processor 305 at step 905 is not an access request, the method continues at step 920. Otherwise, the method continues at step 930.

At step 920 the processor 305 performs a test to determine if the request received by the processor 305 at step 905 is a share request. Upon a determination by the processor 305 at step 920 the request received by the processor 305 at step 905 is not a share request, the method continues at step 925. Otherwise, the method continues at step 930.

At step 925 the processor 305 performs a test to determine if the request received by the processor 305 at step 905 is a revocation request. Upon a determination by the processor 305 at step 925 the request received by the processor 305 at step 905 is not a revocation request, the method continues at step 905. The processor 305 may determine the request is a revocation request based on user input. Otherwise, the method continues at step 950.

At step 930 the processor 305 performs a test to determine if access to the patient's data should be granted, based on the patient giving consent to access the patient's medical data controlled by the patient using the patient's health record storage account secured by the PHR platform 120. Upon a determination by the processor 305 at step 930 that the request for access to the patient's data should not be granted, the method continues at step 945. Upon a determination by the processor 305 at step 930 that the request for access to the patient's data should be granted, the method continues at step 935.

At step 935, the processor 305 determines the access duration. The processor 305 may determine the access duration based on user input, user preference determined from user account profile data, or other factors. The method continues at step 940.

At step 940, the processor 305 gives consent to the request. The processor 305 may give consent to the requestor based on receiving the patient's consent to access the patient's data. The processor 305 at step 940 may give consent based on sending a digital message to the PHR platform 120, to another patient device, or to a provider device, as herein disclosed. The processor 305 at step 940 may give consent based on updating state, or doing nothing. In any case, the method continues at step 905.

At step 945, the processor 305 denies access. The processor 305 may deny access to the requestor based on receiving the patient's refusal to grant access to the patient's data. The processor 305 at step 945 may deny access based on sending a digital message to the PHR platform 120, to another patient device, or to a provider device, as herein disclosed. The processor 305 at step 945 may deny access based on updating state, or doing nothing. In any case, the method continues at step 905.

At step 950, the processor 305 revokes access. The processor 305 may revoke access by the requestor to the patient's data based on the processor 305 determining the requestor should not have further access to the patient's data. The processor 305 at step 950 may revoke access based on sending a digital message to the PHR platform 120, updating state, or doing nothing. In any case, the method continues at step 905.

In some implementations, the method may repeat. In various implementations, the method may end.

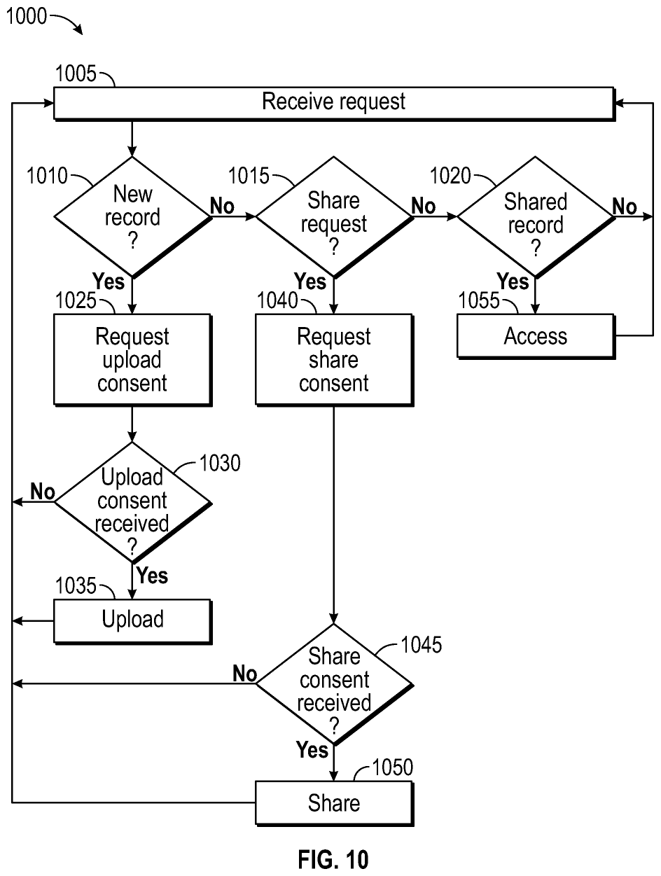

FIG. 10 depicts a process flow of an exemplary PHRAE provider aspect implementation configured to provide secure access to a patient's stored health record data, in response to receiving the patient's consent to access the data. In FIG. 10, the depicted method is given from the perspective of the Personal Health Record Access Engine (PHRAE) 325 implemented via processor-executable program instructions executing on a processor configured in the provider A device 110, depicted at least in FIG. 1 and FIG. 2. To facilitate efficient description, the processor 305, depicted in FIG. 3, is identified for this example as representative of the provider A device 110 processor. In various implementations, the method depicted in FIG. 10 may also be understood by one of ordinary skill as if given from the perspective of the provider B device 130, implemented via processor-executable program instructions executing on a processor configured in the provider B device 130, depicted at least in FIG. 1 and FIG. 2. In the illustrated implementation and using FIG. 3 as representative of the provider A device 110 or the provider B device 130 depending on the aspect disclosed, the PHRAE 325 executes as program instructions on the processor 305 configured in the PHRAE 325 host provider A device 110, depicted in at least FIG. 1 and FIG. 2. In some implementations, the PHRAE 325 may execute as a cloud service communicatively and operatively coupled with system services, hardware resources, or software elements local to and/or external to the PHRAE 325 host provider A device 110.

In an illustrative example, the PHRAE 325 implementation executing as program instructions on the processor 305 may be designed with event-driven or interrupt-driven embedded programming techniques to detect and process events based on received messages, captured sensor data, user input, or other event triggers as would be known to one of ordinary skill in the arts of embedded control systems. Some PHRAE 325 implementations may designate various events as having different priorities, as a design decision within the scope of one having ordinary skill in the art. Various exemplary PHRAE 325 implementations may process events having different priorities in an order different from the order in which the events were detected or different from the order in which the events occurred, based on configured event priority determined by a designer having ordinary skill in the art. In an illustrative example, the exemplary PHRAE 325 process flow given with reference to the drawing represents an example of real-time asynchronous event handling, and an exemplary PHRAE 325 process implementation may handle events in a different order as a result of real-time conditions, design decisions, and other factors, as the skilled artisan would recognize. For example, an exemplary PHRAE 325 process may prioritize an event related to user privacy or data security for handling before an event related to file system maintenance.

The depicted method 1000 begins at step 1005 with the processor 305 receiving a request. The request may be a request related to access to the patient's medical data. For example, the request received by the processor 305 at step 1005 may include a new health record, a request for the patient to provide consent for the requestor to share the patient's medical data using the patient's health record storage account secured by the PHR platform 120, or the request may be a patient health record shared from another provider. The method continues at step 1010

At step 1010 the processor 305 performs a test to determine if the request received by the processor 305 at step 1005 is a new health record. The new health record may be a result of a provider request to upload medical data generated by the provider. Upon a determination by the processor 305 at step 1010 the request received by the processor 305 at step 1005 is not a new health record, the method continues at step 1015. Upon a determination by the processor 305 at step 1010 the request received by the processor 305 at step 1005 is a new health record, the method continues at step 1025.

At step 1015 the processor 305 performs a test to determine if the request received by the processor 305 at step 1005 is a share request. The share request may be a result of a provider request to share the patient's medical data with another provider. Upon a determination by the processor 305 at step 1015 the request received by the processor 305 at step 1005 is not a share request, the method continues at step 1020. Upon a determination by the processor 305 at step 1015 the request received by the processor 305 at step 1005 is a share request, the method continues at step 1040.

At step 1020 the processor 305 performs a test to determine if the request received by the processor 305 at step 1005 is a shared record. The shared record may be a result of a provider sharing the patient's medical data. Upon a determination by the processor 305 at step 1020 the request received by the processor 305 at step 1005 is not a shared record, the method continues at step 1005. Upon a determination by the processor 305 at step 1020 the request received by the processor 305 at step 1005 is a shared record, the method continues at step 1055.

At step 1025, the processor 305 requests upload consent. The processor 305 may request upload consent based on sending a digital message including a request for the patient to give consent for access to the patient's health record storage account secured by the PHR platform 120. The method continues at step 1030.

At step 1030, the processor 305 performs a test to determine if upload consent has been received. The processor 305 may determine if upload consent has been received based on processing a digital message received from a patient device. Upon a determination by the processor 305 at step 1030 that upload consent has been received, the method continues at step 1035 with the processor 305 uploading the health record. Otherwise, the method continues at step 1005.

At step 1040, the processor 305 requests share consent. The processor 305 may request share consent based on sending to a patient device a digital message including a request to share the patient's data. The method continues at step 1045.

At step 1045, the processor 305 performs a test to determine if share consent has been received, in response to the share consent requested by the processor 305 at step 1040. Upon a determination by the processor 305 at step 1045 that share consent was not received, the method continues at step 1005. Upon a determination by the processor 305 at step 1045 share consent was received, the method continues at step 1050 with the processor 305 sharing the data based on the patient's consent received by the processor 305 at step 1045. The processor 305 may share the data based on sending a digital message including the data to the provider for which the patient consented to share access to the patient's data.

At step 1055, the processor 305 accesses the shared record included with the request received by the processor 305 at step 1005, and the method continues at step 1005.

In some implementations, the method may repeat. In various implementations, the method may end.

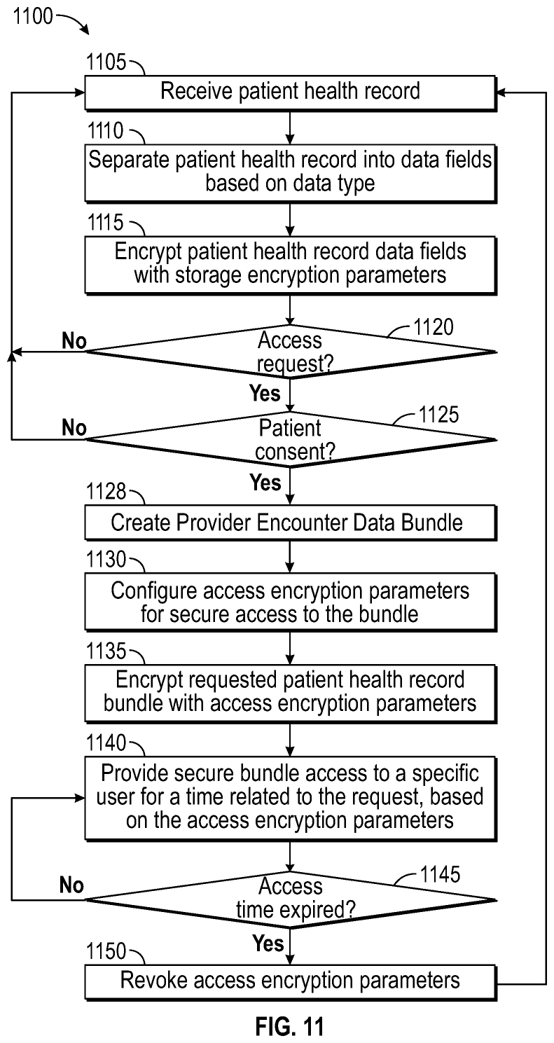

FIG. 11 depicts a process flow of an exemplary PHRAE system aspect configured to provide secure access to a patient's stored health record data, in response to receiving the patient's consent to access the data. In FIG. 11, the depicted method is given from the perspective of the Personal Health Record Access Engine (PHRAE) 325 implemented via processor-executable program instructions executing on the PHR platform 120 processor 305, depicted in FIG. 3. In various implementations, the method depicted in FIG. 11 may also be understood by one of ordinary skill as if given from the perspective of the provider A device 110, the provider B device 130, or the patient device 140, implemented via processor-executable program instructions executing on a processor configured in the respective device. To facilitate efficient disclosure, the processor 305, depicted in FIG. 3, may be understood as representative of the processor configured in the provider A device 110, the provider B device 130, or the patient device 140. The provider A device 110, the provider B device 130, and the patient device 140 are depicted at least in FIG. 1 and FIG. 2. In the illustrated implementation, the PHRAE 325 executes as program instructions on the processor 305 configured in the PHRAE 325 host PHR platform 120, depicted in at least FIG. 1, FIG. 2, and FIG. 3. In some implementations, the PHRAE 325 may execute as a cloud service communicatively and operatively coupled with system services, hardware resources, or software elements local to and/or external to the PHRAE 325 host PHR platform 120.

In an illustrative example, the PHRAE 325 implementation executing as program instructions on the processor 305 may be designed with event-driven or interrupt-driven embedded programming techniques to detect and process events based on received messages, captured sensor data, user input, or other event triggers as would be known to one of ordinary skill in the arts of embedded control systems. Some PHRAE 325 implementations may designate various events as having different priorities, as a design decision within the scope of one having ordinary skill in the art. Various exemplary PHRAE 325 implementations may process events having different priorities in an order different from the order in which the events were detected or different from the order in which the events occurred, based on configured event priority determined by a designer having ordinary skill in the art. In an illustrative example, the exemplary PHRAE 325 process flow given with reference to the drawing represents an example of real-time asynchronous event handling, and an exemplary PHRAE 325 process implementation may handle events in a different order as a result of real-time conditions, design decisions, and other factors, as the skilled artisan would recognize. For example, an exemplary PHRAE 325 process may prioritize an event related to user privacy or data security for handling before an event related to file system maintenance.

The depicted method 1100 begins at step 1105 with the processor 305 receiving a patient health record. The method continues at step 1110.

At step 1110, the processor 305 separates the patient health record into data fields based on data type. The method continues at step 1115.

At step 1115, the processor 305 encrypts the patient heath record data fields with storage encryption parameters. The method continues at step 1120.

At step 1120, the processor 305 performs a test to determine if a request has been received to access the patient's health record data. Upon a determination by the processor 305 at step 1120 that no request has been received to access the patient's health record data, the method continues at step 1105. Upon a determination by the processor 305 at step 1120 that a request has been received to access the patient's health record data, the method continues at step 1125.

At step 1125, the processor 305 performs a test to determine if the patient has given consent to the request for access to the patient's health record data identified by the processor 305 at step 1120. In the depicted implementation, the patient access consent determined by the processor 305 at step 1125 represents the patient granting temporary approval for at least a subset of the patient's EHR data to be pushed to a specific provider, for a near-future healthcare encounter. Upon a determination by the processor 305 at step 1125 that the patient has not given consent to the request for access to the patient's health record data, the method continues at step 1105. Upon a determination by the processor 305 at step 1125 that the patient has given consent to the request for access to the patient's health record data, the method continues at step 1128.

At step 1128, the processor 305 creates a provider encounter data bundle. In the depicted implementation, the data bundle may be created to provide access to all or a subset of the patient's EHR. The processor 305 may create the data bundle with a structure designed to permit individual access to data fields and records included with the patient's EHR. Each data field may be individually protected by SE&KM. The data bundle structure created by the processor 305 may include, for example, various files. The data bundle structure may include various files organized in folders. The method continues at step 1130.

At step 1130, the processor 305 configures access encryption parameters for secure access by the requestor to the bundle created by the processor 305 at step 1128. The method continues at step 1135.

At step 1135, the processor 305 encrypts the requested patient health record bundle with the access encryption parameters. The encrypted stored data may be decrypted to clear-text, and the clear-text then encrypted using a per request session key, suitable for protection-in-transit. The method continues at step 1140.

At step 1140, the processor 305 provides secure bundle access to the requestor for a time related to the request, based on the access encryption parameters. When in-transit protected data arrives at the requestor device, transport encryption may be removed, and SE&KM may be applied for data-at-rest protection. The method continues at step 1145.

At step 1145, the processor 305 performs a test to determine if access time has expired. Upon a determination by the processor 305 at step 1145 access time for the requestor has expired, the method continues at step 1150. Upon a determination by the processor 305 at step 1145 access time for the requestor has not expired, the method continues at step 1140.

At step 1150, the processor 305 revokes the access encryption parameters, and the method continues at step 1105.

In some implementations, the method may repeat. In various implementations, the method may end.

Although various features have been described with reference to the Figures, other features are possible. For example, various implementations may be referred to as MyMR. A MyMR implementation may provide a PHR platform configured to permit patients to own and control the flow of their medical records, as the medical records are produced, collected, organized, and shared between patients and providers.

Some MyMR implementations may empower a patient as the owner and guardian of their health records, permitting the patient to govern collection and exchange of the patient's health-related data.

Various MyMR implementations may provide a cloud-based PHR platform that lets patients collect, store, and control their medical records. An exemplary MyMR implementation may connect patients with providers, payers, and hospitals, providing software to collect, organize, view, and share patient medical record data seamlessly as the patient chooses. The platform may be configured to permit medical and health information upload and download to and from providers based on patient consent. The data security is based on SE&KM, a highly sophisticated proprietary encryption technique with key management tools that allow the patient to control the flow of data. Various MyMR implementations may be configured to share medical records. Some MyMR implementations may be configured to provide additional medical-related services, based on leveraging networks of providers and doctors.

In an exemplary MyMR implementation usage scenario, a patient may be a recipient of a medical service from a provider or hospital. The MyMR implementation may permit the patient to control the flow of medical records in and out of the patient's secure storage configured in a MyMR implementation.

In an exemplary MyMR implementation usage scenario, a provider may be a physical therapist, home health care company, medical equipment company, laboratory, pharmacy, imaging facility, therapist, outpatient surgery clinic, urgent care center, or hospital providing care to a patient.

In an exemplary MyMR implementation, a patient may give consent to a provider, to upload medical records into the patient's medical record storage secured by MyMR. The patient may then use the MyMR platform to give consent to share the medical records. The medical records the patient has given consent to share may then be shared with any other provider having MyMR software installed in their devices. The patient controls their records, and the patient may share the medical records with any provider or doctor. The patient may also decide the duration of the access that each provider can have.

An exemplary MyMR implementation may be a web-based application across all user levels. In an illustrative example, a MyMR implementation may store collected data in a cloud database. An exemplary cloud architecture may permit a MyMR implementation access to files and functionalities from anywhere, independent of location, based on internet-connected and web-enabled devices configured with web browsing capability. An exemplary MyMR implementation may collect, store, and configure secure access to data from all user levels. Various different kinds of data, such as documents, images, metadata, or media files, may be stored.

A patient's medical data generated by a provider or doctor may be transferred to the patient's medical record storage secured by MyMR upon the patient's request. In an illustrative example, when the patient's medical data reaches the patient's medical record storage secured by MyMR, the patient's medical records secured by MyMR are then officially owned by the patient. An exemplary MyMR implementation may provide the patient with the ability to exercise the patient's right to assert ownership control of the patient's data, including the right of the patient to do anything with their data as they so desire. Accordingly, an exemplary MyMR implementation may facilitate the storage, access, exchange, or deletion of the patient's data, based on the patient's consent.

In some MyMR implementations, if the sender of a medical record includes another provider as a recipient, the recipient may be notified of the incoming medical record. The recipient then has multiple ways to access the medical record, including 1) By downloading from the incoming notification; 2) Login to the portal and download the document; or, 3) Using a macro.

In an illustrative example, the HIPAA (Health Insurance Portability and Accountability Act) requires the consent of the patient for the providers to send medical records out of the provider's facility. Patient consent may be obtained by having a patient sign a medical record disclosure form. Once patient consent is received by the provider, the provider may upload the patient's medical records to the patient's medical record storage secured by MyMR. The provider upload process may be achieved with the provider logged in to a MyMR portal, using the proprietary MyMR workflow mechanism based on uploading a document into the MyMR database. The document may be a PDF document visually rendered by a PDF File Generator process. The rendered document may be processed by AI technology to extract data from the rendered document. The extracted data may be input to an EHR by a macro configured to search the extracted data for information required by the EHR, and input the data to the EHR. The EHR encoding the data extracted from the rendered document may be input to the MyMR system. The provider may enter the patient's information, and initiate the upload. The file is then encrypted using SE&KM, and stored in the patient's MyMR account.

Some MyMR implementations may be configured with a pull mechanism for the data using structured formats, such as, for example, HL7 (FHIR). An exemplary MyMR implementation may be configured to use biometrics to authenticate the patient, provide appropriate consent, and initiate data transactions through mobile or web applications from the patient's side, to pull data that resides in provider's electronic records. In an illustrative example, once the records are pulled, the records may be organized and stored in folders accessible to the patient accounts. The patient will have access to the folders using personal devices (for example a mobile phone app configured to implement an interface to MyMR services) and can transmit or share any or all documents to anyone they choose, and specify view or download privileges, duration, and conditions. The web and mobile app may also provide a tool to create a patient ‘avatar’ summarizing current medical data. A provider may be equipped with a mobile phone app configured to implement an interface to MyMR services from the perspective of a provider.

In an illustrative example, the data input into a MyMR implementation may be in a non-structured format, such as PDF or XML. The data input into a MyMR implementation may be in a structured format, such as HL7, FHIR, or the like. The data input may be interpreted by AI. First, the patient account may be verified. Patient account verification may be based on at least 3 demographic or account characteristics (for example, MyMR number) matches. The data may then be split into files and stored into folders accessible in the account. The system may also use AI to synthesize and organize the data in easily viewable formats, such as, for example, text documents, pictures, movies, and an individual ‘avatar’ that represents available data. Some MyMR implementations may also use AI to scan unstructured data to create structured data that may be more useful to a patient and the providers. AI may also be used to identify actionable health information for the patients and provide links and resources identified to the patient, enabling the patient to take appropriate action more quickly. In an implementation example, AI may be configured in a MyMR system to analyze data in selected anonymized datasets, to help with research, insurance guidance, targeted health advertising, and population health.

In an exemplary implementation, MyMR may be configured with software such as, for example, PDF File Generator software, which may be downloaded in the provider's computer. The provider then may submit any requested patient document from their electronic health records directly into MyMR cloud accounts in a PDF format. Similarly, a download tool software may reside in the provider's computer which initially notifies the provider that a document has been sent to them and is waiting to be viewed or downloaded. Once the provider clicks the notification, a macro set may open up automatically, to help the provider to view, select, or download the records into any folders they choose.

In various exemplary MyMR implementations, encrypted stored data may be decrypted to clear-text, and the clear-text then encrypted using a per request session key, suitable for protection-in-transit. Conversely, when in-transit protected data arrives from the client to the server-side application, transport encryption is removed, and then SE&KM applied for data-at-rest protection.

The mechanism of SE&KM allows secure transfer of data over the internet. Every bit of data transferred via a MyMR implementation may be configured to use SE&KM technology that provides the highest level of encryption to protect the sensitive medical data.

In an illustrative example, users of MyMR may include patients, providers, and doctors, exchanging and transferring medical records of different sizes. For example, a medical record may be a PDF document of many pages including image files such as X-rays, notes, prescriptions, and the like.

A MyMR implementation makes patient medical records available to a provider governed by patient consent. A patient may log in, select the folders they want to share, enter the recipient details, and initiate the transfer. The records are then made available to the recipient. The recipient may be a provider. The records may be made available to the provider for download.

In the Summary above and in this Detailed Description, and the Claims below, and in the accompanying drawings, reference is made to particular features of various implementations. It is to be understood that the disclosure of particular features of various implementations in this specification is to be interpreted to include all possible combinations of such particular features. For example, where a particular feature is disclosed in the context of a particular aspect or implementation, or a particular claim, that feature can also be used—to the extent possible—in combination with and/or in the context of other particular aspects and implementations, and in an implementation generally.

While multiple implementations are disclosed, still other implementations will become apparent to those skilled in the art from this detailed description. Disclosed implementations may be capable of myriad modifications in various obvious aspects, all without departing from the spirit and scope of the disclosed implementations. Accordingly, the drawings and descriptions are to be regarded as illustrative in nature and not restrictive.

It should be noted that the features illustrated in the drawings are not necessarily drawn to scale, and features of one implementation may be employed with other implementations as the skilled artisan would recognize, even if not explicitly stated herein. Descriptions of well-known components and processing techniques may be omitted so as to not unnecessarily obscure the implementation features.

In the present disclosure, various features may be designated as optional/noncompulsory, for example, through the use of the verb “may;” or, through the use of any of the phrases: “in some implementations,” “in some designs,” “in various implementations,” “in various designs,” “in an illustrative example,” or, “for example.” In other words, something designated optional/noncompulsory can, but need not. In other words, something that “may” can, but need not. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be implemented in seven different ways, namely with just one of the three possible features, with any two of the three possible features or with all three of the three possible features.

In various implementations, elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various implementations have been disclosed and described in detail herein, it will be apparent to those skilled in the art that various changes may be made to the disclosed configuration, operation, and form without departing from the spirit and scope thereof. In particular, it is noted that the respective implementation features, even those disclosed solely in combination with other implementation features, may be combined in any configuration excepting those readily apparent to the person skilled in the art as nonsensical. Likewise, use of the singular and plural is solely for the sake of illustration and is not to be interpreted as limiting.

The Abstract is provided to comply with 37 C. F. R. § 1.72(b), to allow the reader to quickly ascertain the nature of the technical disclosure and is submitted with the understanding that it will not be used to interpret or limit the scope or meaning of the claims.

In the present disclosure, all descriptions where “comprising” is used may have as alternatives “consisting essentially of,” or “consisting of.” In the present disclosure, any method or apparatus implementation may be devoid of one or more process steps or components. In the present disclosure, implementations employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an implementation “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of implementation apparatus are known in the art. One or more implementation part may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The terms “abutting” or “in mechanical union” refer to items that are in direct physical contact with each other, although the items may not necessarily be attached together.

The word “exemplary” is used herein to mean “serving as an example, instance, or illustration.” Any implementation described herein as “exemplary” is not necessarily to be construed as preferred over other implementations. While various aspects of the disclosure are presented with reference to drawings, the drawings are not necessarily drawn to scale unless specifically indicated.

Reference throughout this specification to “an implementation” or “the implementation” means that a particular feature, structure, or characteristic described in connection with that implementation is included in at least one implementation. Thus, the quoted phrases, or variations thereof, as recited throughout this specification are not necessarily all referring to the same implementation.

Similarly, it should be appreciated that in the above description, various features are sometimes grouped together in a single implementation, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects may lie in a combination of fewer than all features of any single foregoing disclosed implementation. Thus, the claims following this Detailed Description are hereby expressly incorporated into this Detailed Description, with each claim standing on its own as a separate implementation. This disclosure is intended to be interpreted as including all permutations of the independent claims with their dependent claims.

Various system and method implementations in accordance with the present disclosure may be accomplished through the use of one or more computing devices. As depicted, for example, at least in FIG. 1, FIG. 2, and FIG. 3, one of ordinary skill in the art would appreciate that an exemplary system appropriate for use with implementation in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with implementations in accordance with the present disclosure include, but are not limited to, proprietary computing devices, personal computers, mobile computing devices, tablet PCs, mini-PCs, servers, or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and implementation of the present disclosure are contemplated for use with any computing device.

In various implementations, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with implementations of the present disclosure, and implementations of the present disclosure are contemplated for use with any appropriate configuration.

According to an implementation of the present disclosure, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth® connections, or any combination thereof. One of ordinary skill in the art would appreciate that there are numerous communications means that may be utilized with implementations of the present disclosure, and implementations of the present disclosure are contemplated for use with any communications means.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an implementation may include an alternate order of the steps adapted to a particular application of a technique disclosed herein. All such variations and modifications are intended to fall within the scope of this disclosure. The depiction and description of steps in any particular order is not intended to exclude implementations having the steps in a different order, unless required by a particular application, explicitly stated, or otherwise clear from the context.

Traditionally, a computer program consists of a sequence of computational instructions or program instructions. It will be appreciated that a programmable apparatus (that is, computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable, and replaceable, or fixed. It will also be understood that a computer can include a Basic Input/Output System (BIOS), firmware, an operating system, a database, or the like that can include, interface with, or support the software and hardware described herein.

Implementations of the system as described herein are not limited to applications involving conventional computer programs or programmable apparatuses that run them. It is contemplated, for example, that implementations of the disclosure as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code encoded therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code encoded by a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, implementations that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, implementations of the disclosure are not described with reference to any particular programming language. It is appreciated that a variety of programming languages may be used to implement the present teachings as described herein, and any references to specific languages are provided for disclosure of enablement and best mode of implementations of the disclosure. Implementations of the disclosure are well suited to a wide variety of computer network systems over numerous topologies. Within this field, the configuration and management of large networks include storage devices and computers that are communicatively coupled to dissimilar computers and storage devices over a network, such as the Internet.

The respective reference numbers and descriptions of certain of the elements depicted by the Drawings are summarized as follows.
105 provider
110 provider A device
115 network cloud
120 PHR platform
125 storage encryption parameters
130 provider B device
135 patient
140 patient device
145 access encryption parameters
201 wireless access point
202 wireless link
203 router
204 communication link
205 communication link
206 wireless access point
207 wireless communication link
305 processor
310 memory
315 program memory
320 data memory
325 PHRAE (Personal Health Record Access Engine)
330 storage medium
335 I/O interface
340 user interface
345 multimedia interface
400 PHR sharing scenario
500 PHR process flow
505 provider A device upload
510 PHR platform device store
515 patient device consent
520 provider B device access
525 provider A upload
530 patient records
535 patient share consent
600 PHR data security implementation
700 PHR data flow
705 provider A device data flow
710 PHR platform device data flow
715 patient device data flow
720 provider B device data flow
725 medical record document
730 pharmacy documents
735 patient consent
800 PHRAE platform aspect process flow
900 PHRAE patient aspect process flow
1000 PHRAE provider aspect process flow
1100 PHRAE system aspect process flow

A number of implementations have been described. Nevertheless, it will be understood that various modifications may be made. For example, the steps of the disclosed techniques may be performed in a different sequence, components of the disclosed systems may be combined in a different manner, or the components may be supplemented with other components. Accordingly, other implementations are contemplated, within the scope of the following claims.

1: An apparatus comprising:
a processor; and
a memory that is not a transitory propagating signal, the memory configured to be operably connected to the processor and including computer readable instructions, including processor executable program instructions, the computer readable instructions accessible to the processor, wherein the processor executable instructions, when executed by the processor, cause the processor to perform operations comprising:
store a patient's health record data encrypted with storage encryption parameters; and
in response to the patient giving consent to a data access request:
configure secure access to the patient's health record data based on encrypting the stored data with access encryption parameters distinct from the storage encryption parameters; and
provide the secure access to a specific device at a location and time related to the request.

2: The apparatus of claim 1, wherein the data access request further comprises a data upload operation request.

3: The apparatus of claim 1, wherein the data access request further comprises a data download operation request.

4: The apparatus of claim 1, wherein the data access request further comprises a data share operation request.

5: The apparatus of claim 1, wherein the data access request further comprises a data delete operation request.

6: The apparatus of claim 2, wherein provide the secure access further comprises implement the data operation request.

7: The apparatus of claim 1, wherein store the patient's health record data further comprises separate the health record data into a plurality of data fields based on the data type.

8: The apparatus of claim 7, wherein store the patient health record data further comprises encrypt each data field of the plurality of data fields with a storage encryption parameter comprising an encryption key uniquely determined as a function of information related to the patient.

9: The apparatus of claim 7, wherein configure secure access further comprises configure distinct access encryption parameters for each data field of the plurality of data fields.

10: The apparatus of claim 7, wherein the access encryption parameters further comprise a decryption key uniquely determined as a function of an individual data field.

11: The apparatus of claim 1, wherein the access encryption parameters further comprise a decryption key uniquely determined as a function of the data access request.

12: The apparatus of claim 11, wherein provide the secure access further comprises sending a digital message comprising the access encryption parameters.

13: The apparatus of claim 12, wherein the digital message further comprises an encrypted reference to the decryption key.

14: The apparatus of claim 1, wherein provide the secure access further comprises send a digital message comprising a response to the data access request.

15: The apparatus of claim 14, wherein the response further comprises consent to the data access request.

16: The apparatus of claim 14, wherein the response further comprises denial to the data access request.

17: The apparatus of claim 14, wherein the response is determined as a function of the data access requestor's identity authenticated based on sensor data.

18: The apparatus of claim 14, wherein the response is determined as a function of the data access requestor's location authenticated based on sensor data.

19: The apparatus of claim 1, wherein the operations performed by the processor further comprise in response to a patient withdrawing consent to data access, revoking the configured access to the data.

20: The apparatus of claim 1, wherein the operations performed by the processor further comprise in response to a patient giving consent to a data access request, creating a provider encounter data bundle.

21: The apparatus of claim 20, wherein the provider encounter data bundle structure is designed to permit individual access to patient data fields and records.

22: An apparatus comprising:
a processor;
a cloud database, operably coupled with the processor; and
a memory that is not a transitory propagating signal, the memory configured to be operably connected to the processor and including computer readable instructions, including processor executable program instructions, the computer readable instructions accessible to the processor, wherein the processor executable instructions, when executed by the processor, cause the processor to perform operations comprising:
receive a digital message comprising a request by a provider to upload a patient's health record data;
send a digital message comprising a request for the patient's consent for the provider to upload the health record data; and
in response to receiving a digital message comprising the patient's consent to upload the health record data:
send a digital message to the provider comprising consent to upload the health record data;
receive a digital message comprising the health record data;
separate the health record data into a plurality of data fields based on the data type determined for each data field of the plurality of data fields;
encrypt each data field of the plurality of data fields based on storage encryption parameters comprising an encryption key uniquely determined as a function of account information associated to the patient; and
store the encrypted health record data to the cloud database.

23: The apparatus of claim 22, wherein separate the health record data further comprises digitally rendering the health record data based on printing the health record data to a file.

24: The apparatus of claim 23, wherein separate the health record data further comprises extract to digital form the data encoded by the rendered health record.

25: The apparatus of claim 22, wherein separate the health record data further comprises the data type determined by an AI (Artificial Intelligence) process configured to recognize the type of data encoded by a health record data field.

26: The apparatus of claim 22, wherein the digital message comprising the request for the patient's consent further comprises the provider location authenticated based on sensor data received from the provider.

27: The apparatus of claim 22, wherein the digital message comprising the request for the patient's consent further comprises the provider device authenticated based on a communication interface parameter received from the provider.

28: The apparatus of claim 22, wherein the operations performed by the processor further comprise in response to receiving the digital message comprising the patient's consent, creating a provider encounter data bundle.

29: The apparatus of claim 28, wherein the provider encounter data bundle structure is designed to permit individual access to patient data fields and records.

30-42. (canceled)

43: An apparatus comprising:
a processor; and
a memory that is not a transitory propagating signal, the memory configured to be operably connected to the processor and including computer readable instructions, including processor executable program instructions, the computer readable instructions accessible to the processor, wherein the processor executable instructions, when executed by the processor, cause the processor to perform operations comprising:
in response to receiving a request to download a patient's stored health record data encrypted with storage encryption parameters:
determine if the patient has given consent for the requestor to download the patient's health record data; and
in response to determining the patient has given consent:
configure secure access by the requestor to the patient's health record data, based on providing the requestor access encryption parameters distinct from the storage encryption parameters; and
provide the secure access to the requestor for a specific device at a location and time related to the request; and
in response to determining the patient has not given consent:
deny the request.

44: The apparatus of claim 43, wherein the apparatus further comprises a cloud database operably coupled with the processor, and provide the secure access further comprises provide the secure access to the health record data by the database.

45: The apparatus of claim 43, wherein receiving the request to download the patient's health record data further comprises receiving a digital message comprising the request.

46: The apparatus of claim 43, wherein determine if the patient has given consent further comprises determine if the patient's consent is predetermined for the requestor.

47: The apparatus of claim 43, wherein determine if the patient has given consent further comprises send a digital message comprising a request for the patient's consent.

48: The apparatus of claim 47, wherein determine if the patient has given consent further comprises receive a digital message comprising the patient's consent.

49: The apparatus of claim 43, wherein the health record data further comprises a plurality of data fields.

50: The apparatus of claim 43, wherein the access parameters further comprise an encrypted reference to a unique session key configured to secure the health record data in transit.

51: The apparatus of claim 43, wherein provide the secure access to the requestor for a specific device at a location and time related to the request further comprises the device authenticated based on a security certificate.

52: The apparatus of claim 43, wherein provide the secure access to the requestor for a specific device at a location and time related to the request further comprises the location determined based on sensor data.

53: The apparatus of claim 43, wherein provide the secure access to the requestor for a specific device at a location and time related to the request further comprises the access time limited based on the access parameters configured to expire at a predetermined time.

54: The apparatus of claim 43, wherein deny the request further comprises send a digital message comprising request denial.

55: A method comprising:
storing to a cloud database a patient's health record data encrypted with storage encryption parameters; and
in response to the patient giving consent from a mobile device to a data access request by a provider:
configuring secure access to the patient's health record data stored by the cloud database, based on encrypting the stored data with access encryption parameters distinct from the storage encryption parameters; and
providing the secure access to the health record data stored by the cloud database to a specific provider device at a location and time related to the request.

56: The method of claim 55, wherein the data access request further comprises a data upload operation request.

57: The method of claim 55, wherein the data access request further comprises a data download operation request.

58: The method of claim 55, wherein the data access request further comprises a data share operation request.

59: The method of claim 55, wherein the data access request further comprises a data delete operation request.

60: The method of claim 56, wherein providing the secure access further comprises implementing the data operation request.

61: The method of claim 55, wherein storing the patient's health record data further comprises separate the health record data into a plurality of data fields based on the data type.

62: The method of claim 61, wherein storing the patient health record data further comprises encrypt each data field of the plurality of data fields with a storage encryption parameter comprising an encryption key uniquely determined as a function of information related to the patient.

63: The method of claim 61 wherein configuring secure access further comprises configure distinct access encryption parameters for each data field of the plurality of data fields.

64: The method of claim 61, wherein the access encryption parameters further comprise a decryption key uniquely determined as a function of an individual data field.

65: The method of claim 55, wherein the access encryption parameters further comprise a decryption key uniquely determined as a function of the data access request.

66: The method of claim 65, wherein providing the secure access further comprises sending a digital message comprising the access encryption parameters.

67: The method of claim 66, wherein the digital message further comprises an encrypted reference to the decryption key.

68: The method of claim 55, wherein providing the secure access further comprises send a digital message comprising a response to the data access request.

69: The method of claim 68, wherein the response further comprises consent to the data access request.

70: The method of claim 68, wherein the response further comprises denial to the data access request.

71: The method of claim 68, wherein the response is determined as a function of the data access requestor's identity authenticated based on sensor data.

72: The method of claim 68, wherein the response is determined as a function of the data access requestor's location authenticated based on sensor data.

73: The method of claim 55, wherein the operations performed by the processor further comprise in response to a patient withdrawing consent to data access, revoking the configured access to the data.
